(19) United States
(12) Patent Application Publication
(10) Pub. No.: US 2004/0248963 A1
(43) Pub. Date: Dec. 9, 2004

(54) Prodrugs of excitatory amino acids

(76) Inventors: Ana Belen Bueno Melendo, Madrid (ES); Alfonso De Dios, Madrid (ES); Carmen Dominguez-Fernandez, Madrid (ES); Mark Francis Herin, Perwez (BE); Luisa Maria Martin-Cabrejas, Madrid (ES); Jose Alfredo Martin, Madrid (ES); Maria Angeles Martinez-Grau, Madrid (ES); Steven Marc Massey, Indianapolis, IN (US); James Allen Monn, Indianapolis, IN (US); Carlos Montero Salgado, Madrid (ES); Concepcion Pedregal-Tercero, Madrid (ES); Matthew John Valli, Zionsville, IN (US)

Correspondence Address: Arvie J Anderson, Eli Lilly & Company, Patent Division, PO Box 6288, Indianapolis, IN, 46206-6288, US

(21) Appl. No.: 10/494,189

(22) PCT Filed: Nov. 12, 2002
(86) PCT No.: PCT/US02/33626

(30) Foreign Application Priority Data
Nov. 23, 2001 (ES) 01500276.9

(51) Int. Cl. C07D 317/08; A61K 31/4035; A61K 31/195

(52) U.S. Cl. 514/417; 514/561; 548/473; 549/229; 562/501; 514/463

(57) ABSTRACT

This invention relates to synthetic excitatory amino acid prodrugs of formula (I) wherein the residues R¹¹, R¹² and R¹³ are as defined in claim 1. The invention further relates to processes for the preparation of the compounds of formula (I), and to pharmaceutical compositions for the treatment of neurological and psychiatric disorders comprising said compounds.

This invention relates to synthetic excitatory amino acid prodrugs (and their pharmaceutically acceptable salts) and processes for their preparation. The invention further relates to methods of using, and pharmaceutical compositions comprising, the compounds for the treatment of neurological disorders and psychiatric disorders.

Treatment of neurological or psychiatric disorders, such as anxiety disorder, have been linked to selective activation of metabotropic excitatory amino acid receptors such as (+)-2-aminobicyclo[3.1.0]hexane-2,6-dicarboxylic acid, also known as LY354740, which is disclosed in U.S. Pat. No. 5,750,566 (the '566 patent) issued May 12, 1998 is an active mGluR2 receptor agonist. CNS Drug Reviews, 5, pgs. 1-12 (1999).

The present invention provides for a prodrug form of LY354740 which enhances the oral exposure of LY354740. The present invention also provides for prodrug forms of other compounds which possess improved oral exposure. Compounds of the present invention represent an improved approach for maintaining LY354740-like safety and efficacy in humans with increased oral bioavailability. Preclinical studies with compounds of the present invention have shown greatly enhanced oral exposure of the parent compound.

Accordingly, the present invention provides a compound of the formula I

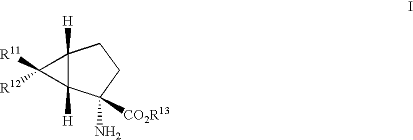

wherein

R¹¹ is CO₂R¹⁴ and R¹² is hydrogen or fluoro; or R¹¹ is hydrogen or fluoro and R¹² is CO₂R¹⁴;

R¹³ and R¹⁴ are, independently, hydrogen, —CHR¹⁵O₂CXR¹⁶ or a group selected from 3-phthalidyl or

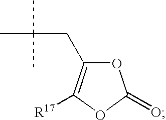

X is O, N, S, or a bond;

R¹⁵ is hydrogen, (1-10C) alkyl, (2-4C) alkenyl, aryl, or arylalkyl;

R¹⁶ is (1-10C) alkyl, (2-4C) alkenyl, (2-4C) alkynyl, or aryl; and

R¹⁷ is hydrogen, (1-10C) alkyl, or phenyl;

provided when R¹⁴ is hydrogen, R¹³ is not hydrogen;

or a pharmaceutically acceptable salt thereof.

Compounds of the present invention have been found to be useful prodrugs for selective agonists of metabotropic glutamate receptors, and are therefore useful in the treatment of diseases of the central nervous system such as neurological diseases, for example neurodegenerative diseases, and as antipsychotic, anxiolytic, drug-withdrawal, antidepressant, anticonvulsant, analgesic and anti-emetic agents.

It will be appreciated that the compounds of formula (I) contain at least four asymmetric carbon atoms, three being in the cyclopropane ring and one being at the α-carbon of the amino acid group within the cyclopentane ring. Additional asymmetric carbons may be present in the generic radicals as defined. Accordingly, the compounds of the invention may exist in and be isolated in enantiomerically pure form, in racemic form, or in a diastereoisomeric mixture.

The amino acid moiety within the cyclopentane ring preferably has the natural amino acid configuration, i.e. the L-configuration relating to D-glyceraldehyde.

The present invention includes pharmaceutically acceptable salts of the compound of formula I. These salts can exist in conjunction with the acidic or basic portion of the molecule and can exist as acid addition, primary, secondary, tertiary, or quaternary ammonium, alkali metal, or alkaline earth metal salts. Generally, the acid addition salts are prepared by the reaction of an acid with a compound of formula I. The alkali metal and alkaline earth metal salts are generally prepared by the reaction of the hydroxide form of the desired metal salt with a compound of formula I.

Acids commonly employed to form such salts include inorganic acids, for example hydrochloric, hydrobromic, nitric, sulphuric or phoshoric acids, or with organic acids, such as organic carboxylic acids, for example, glycollic, maleic, hydroxymaleic, fumaric, malic, tartaric, citric, salicyclic, o-acetoxybenzoic, or organic sulphonic acids, such as 2-hydroxyethane sulphonic, toluene-p-sulphonic, methanesulfonic, ethanesulfonic or naphthalene-2-sulphonic acid.

In addition to pharmaceutically acceptable salts, other salts are included in the invention. They may serve as intermediates in the purification of compounds or in the preparation of other, for example pharmaceutically-acceptable, acid addition salts, or are useful for identification, characterization or purification.

A variety of physiological functions have been shown to be subject to influence by excessive or inappropriate stimulation of excitatory amino acid transmission. The formula I compounds of the present invention are believed to have the ability to treat a variety of neurological disorders in mammals associated with this condition, including acute neurological disorder such as cerebral deficits subsequent to cardiac bypass surgery and grafting, stroke, cerebral ischemia, spinal cord trauma, head trauma, perinatal hypoxia, cardiac arrest, and hypoglycemic neuronal damage. The formula I compounds are believed to have the ability to treat a variety of chronic neurological disorders, such as Alzheimer's disease, Huntington's Chorea, amyotrophic lateral sclerosis, AIDS-induced dementia, ocular damage and retinopathy, cognitive disorders, and idiopathic and drug-induced Parkinson's. The present invention also provides methods for treating these disorders which comprises administering to a patient in need thereof an effective amount of a compound of formula I or a pharmaceutically acceptable salt thereof.

The formula I compounds of the present invention treat a variety of other neurological disorders in mammals that are associated with glutamate dysfunction, including muscular spasms, convulsions, migraine headaches, urinary incontinence, psychosis, (such as schizophrenia), drug tolerance and withdrawal (such as nicotine, opiates and benzodiazepines), anxiety and related disorders, emesis, brain edema, chronic pain, and tardive dyskinesia. The formula I compounds are also useful as antidepressant and analgesic agents. Therefore, the present invention also provides methods for treating these disorders which comprise administering to a patient in need thereof an effective amount of the compound of formula I, or a pharmaceutically acceptable salt thereof.

A compound of formula I may be made by a process which is analogous to one known in the chemical art for the production of structurally analogous heterocyclic compounds or by a novel process described herein. Such processes and intermediates useful for the manufacture of a compound of formula I as defined above are provided as further features of the invention and are illustrated by the following procedures in which, unless otherwise specified, the meanings of the generic radicals are as defined above.

As shown in Scheme 1 below, compounds of formula I are converted via enzymatic or hydrolytic process in vivo, to form compounds of formula III, where R¹³ and R¹⁴ are both hydrogen (a di-acid).

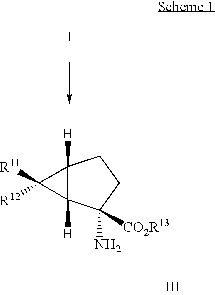

A variety of physiological functions have been shown to be subject to influence by excessive or inappropriate stimulation of excitatory amino acid transmission. The compounds of formula I of the present invention are believed to have the ability to treat a variety of neurological disorders in mammals associated with this condition, including acute neurological disorder such as cerebral deficits subsequent to cardiac bypass surgery and grafting, stroke, cerebral ischemia, spinal cord trauma, head trauma, perinatal hypoxia, cardiac arrest, and hypoglycemic neuronal damage. The compounds of formula I are believed to have the ability to treat a variety of chronic neurological disorders, such as Alzheimer's disease, Huntington's Chorea, amyotrophic lateral sclerosis, AIDS-induced dementia, ocular damage and retinopathy, cognitive disorders, and idiopathic and drug-induced Parkinson's. The present invention also provides methods for treating these disorders which comprises administering to a patient in need thereof an effective amount of a compound of formula I or a pharmaceutically acceptable salt thereof.

The compounds of formula I of the present invention are also believed to have the ability to treat a variety of other neurological disorders in mammals that are associated with glutamate dysfunction, including muscular spasms, convulsions, migraine headaches, urinary incontinence, psychosis, (such as schizophrenia), drug tolerance and withdrawal (such as nicotine, opiates and benzodiazepines), anxiety and related disorders, emesis, brain edema, chronic pain, and tardive dyskinesia. The compounds of formula I are also useful as antidepressant and analgesic agents. Therefore, the present invention also provides methods for treating these disorders which comprise administering to a patient in need thereof an effective amount of the compound of formula I, or a pharmaceutically acceptable salt thereof.

A compound of formula I may be made by a process which is analogous to one known in the chemical art for the production of structurally analogous heterocyclic compounds or by a novel process described herein. Such processes and intermediates useful for the manufacture of a compound of formula I as defined above are provided as further features of the invention and are illustrated by the following procedures in which, unless otherwise specified, the meanings of the generic radicals are as defined above.

(A) For a compound of formula I in which R¹³ is an ester and R¹⁴ is hydrogen, deprotecting the amine protecting group, R^{m}, and the carboxy protecting group, R¹⁴, of a compound of formula II

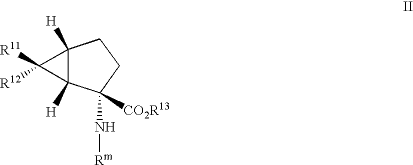

in which R¹³ is an ester, R¹⁴ is a carboxy protecting ester group as described in General Procedure 2 for Examples 13-18.

(B) For a compound of formula II in which R¹³ is an ester, R¹⁴ is a carboxy protecting group, esterifying a compound of formula II where R¹³ is hydrogen and R¹⁴ is a carboxy protecting group as described in General Procedure 1 for Examples 1-6.

(C) For a compound of formula II in which R¹³ is hydrogen and R¹⁴ is not hydrogen, selectively esterifying a compound of formula II in which R¹³ and R¹⁴ are both hydrogen (a di-acid) as described in Example Preparation 1.

(D) For a compound of formula II in which R¹³ and R¹⁴ are both hydrogen (a di-acid), amidating the amine group of a compound of formula I with an amine protecting group as described in Example Preparations 1A and 2.

(E) For a compound of formula I where R¹³ is hydrogen and R¹⁴ is an ester group, deprotecting and ring-opening a compound of formula IV

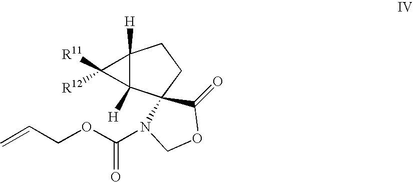

as described in Examples 19-26.

(F) For a compound of formula IV in which R¹⁴ is an ester group, esterifying a compound of formula IV in which R¹⁴ is hydrogen as described in Examples 7-12.

(G) For a compound of formula IV in which R¹⁴ is hydrogen (an acid), protecting and cyclizing a compound of formula III as described in Example Preparation 3.

(H) For a compound of formula I in which-R¹³ and R¹⁴ are not hydrogen (a di-ester), deprotecting the amine group of a compound of formula II as described in General Procedure 5 for Examples 42-56.

(I) For a compound of formula II in which R¹³ and R¹⁴ are not hydrogen (a di-ester), esterifying the carboxy groups of a compound of formula II in which R¹³ and R¹⁴ are both hydrogen (a di-acid) as described in General Procedure 4 for Examples 27-41.

The term “amine protecting group”, as used herein, refers to those groups intended to protect or block the nitrogen group against undesirable reactions during synthetic procedures. Choice of the suitable amine protecting group used will depend upon the conditions that will be employed in subsequent reaction steps wherein protection is required, as is well within the knowledge of one of ordinary skill in the art. Commonly used amine protecting groups are disclosed in T. W. Greene and P. G. M. Wuts, Protective Groups In Organic Synthesis, 3rd Ed. (John Wiley & Sons, New York (1999)). Suitable amine protecting groups comprise acyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, alpha-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like, carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, alpha, alpha-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl and the like; alkyl groups such as benzyl, triphenylmethyl, benzyloxymethyl and the like; and silyl groups such as trimethylsilyl and the like. Preferred suitable amine protecting groups are formyl, acetyl, methyloxycarbonyl, benzoyl, pivaloyl, t-butylacetyl, phenylsulfonyl, benzyl, allyloxycarbonyl, t-butyloxycarbonyl (Boc) and benzyloxycarbonyl (Cbz).

The term “carboxy protecting group” as used herein refers to one of the ester derivatives of the carboxylic acid group commonly employed to block or protect the carboxylic acid group while reactions are carried out on other functional groups of the compound. Particular values of R¹³ and R¹⁴ carboxyl protecting ester groups include, for example, methyl, ethyl, tert-butyl, benzyl, methoxymethyl, trimethylsilyl, allyl and the like. Further examples of such groups may be found in T. W. Greene and P. G. M. Wuts, Protecting Groups in Organic Synthesis, 3^{rd}. Ed. (John Wiley & Sons, N.Y. (1999)). The ester is decomposed by using a conventional procedure which does not affect another portion of the molecule. For example, protecting group removal of a compound of Formula II in which R¹⁴ is allyl using a metal catalyst such as tetrakistriphenylphosphine palladium(0) to produce compounds of formula I where R¹¹ is CO₂R¹⁴, R¹² and R¹⁴ are hydrogen, and R¹³ is —CHR¹⁵O₂CXR¹⁶.

Whereafter, for any of the above procedures, when a pharmaceutically acceptable salt of a compound of formula I is required, it is obtained by reacting the acid of formula I with a physiologically acceptable base or by reacting a basic compound of formula I with a physiologically acceptable acid or by any other conventional procedure.

The term “(1-10C) alkyl” represents a straight, branched, or cyclic alkyl chain having from one to ten carbon atoms. Typical straight or branched (1-10C) alkyl groups include methyl, ethyl, n-propyl, isopropyl, n-butyl, isobutyl, sec-butyl, t-butyl, n-pentyl, isopentyl, neopentyl, n-hexyl, 2-methylpentyl, 3-methylpentyl, 4-methylpentyl, 2,2-dimethylbutyl, 2,3-dimethylbutyl, 3,3-dimethylbutyl, heptyl, n-octyl, 2,2-dimethylhexyl, 2,5-dimethylhexyl, 2-methylheptyl, 4-methylheptyl, 2,2,4-trimethylpentyl, 2,3,4-trimethylpentyl, nonyl, 3,5,5-trimethylhexyl, decyl, 3,7-dimethyloctyl, and the like. The term “(1-10C) alkyl” includes within it the terms “(1-6C) alkyl” and “(1-4C) alkyl”. Typical cyclic alkyl groups include cyclopropyl, cyclobutyl, cyclopentyl, cyclohexyl, cycloheptyl, and the like. Typical (1-6C) alkyl groups include methyl, ethyl, n-propyl, isopropyl, n-butyl, isobutyl, sec-butyl, t-butyl, n-pentyl, isopentyl, neopentyl, and n-hexyl.

The term “1(2-4C) alkenyl” represents straight or branched unsaturated alkyl chains having from two to ten carbon atoms, and having one or more carbon-carbon double bond, such as, dienes. This group also includes both E and Z isomers. Representative radicals for this group include vinyl, allyl, allenyl, 1-butenyl, 2-butenyl, 2-methyl-1-propenyl, 3-butenyl, 2-methyl-2-propenyl, butadienyl, and the like.

The term “(2-4C) alkynyl” means an aliphatic hydrocarbon group containing a carbon-carbon triple bond and which may be straight or branched having about 2 to about 4 carbon atoms in the chain. Branched means that one or more lower alkyl groups such as methyl or ethyl are attached to a linear alkynyl chain.

The term “aryl” represents groups such as phenyl, substituted phenyl, and polycyclic aromatic rings such as 1-naphthyl or 2-naphthyl.

The term “arylalkyl” represents a (1-4C) alkyl group bearing one or more aryl groups. Representatives of this latter group include benzyl, 2-nitrobenzyl, 4-nitrobenzyl, 1-phenylethyl, 2-phenylethyl, 3-phenylpropyl, 4-phenylbutyl, 2-methyl-2-phenylpropyl, (4-chlorophenyl)methyl, (2,6-dichlorophenyl)-methyl, bis(2,6-dichlorophenyl)methyl, (4-hydroxyphenyl)-methyl, (2,4-dinitrophenyl)methyl, triphenylmethyl, (4-methoxyphenyl)diphenylmethyl, bis(4-methoxyphenyl)methyl, α-naphthyldiphenylmethyl, bis(2-nitrophenyl)methyl, and the like.

The term “substituted phenyl,” as used herein, represents a phenyl group substituted with one or two moieties chosen from the group consisting of halogen, hydroxy, cyano, nitro, (1-10C) alkyl, (1-4C) alkoxy, alkoxycarbonyl, protected carboxy, carboxymethyl, hydroxymethyl, amino, protected amino, aminomethyl, or trifluoromethyl. Examples of a substituted phenyl group include 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl, 4-hydroxyphenyl, 3-hydroxy-phenyl, 2,4-dihydroxyphenyl, 3-nitrophenyl, 4-nitrophenyl, 4-cyanophenyl, 4-methylphenyl, 4-ethylphenyl, 4-ethoxy-phenyl, 4-carboxyphenyl, 4-(hydroxymethyl)phenyl, 4-aminophenyl, 4-propylphenyl, 4-butylphenyl, 4-t-butyl-phenyl, 3-fluoro-2-methylphenyl, 2,3-difluorophenyl, 2,6-difluorophenyl, 2,6-dimethylphenyl, 2-fluoro-5-methyl-phenyl, 2,4,6-trifluorophenyl, 2-trifluoromethylphenyl, 2-chloro-5-trifluoromethylphenyl, 2,4-bis(trifluoromethyl)-phenyl, 3,5-bis(trifluoromethyl)phenyl, 2-methoxyphenyl, 3-methoxyphenyl, 4-methoxyphenyl, 3,5-dimethoxyphenyl, 4-hydroxy-3-methyl-phenyl, 3,5-dimethyl-4-hydroxyphenyl, 4-hydroxy-3-(hydroxymethyl)phenyl, 2-amino-5-methylphenyl, 4-amino-3-trifluoromethylphenyl, 3-amino-4-hydroxyphenyl, 2-methyl-4-nitrophenyl, 4-methoxy-2-nitrophenyl, 2,4-dinitrophenyl, 3-cyano-4-nitrophenyl, and the like.

While all the formula I compounds of the present invention are believed to provide prodrug forms of compounds which possess improved oral exposure, certain compounds of the invention are preferred for such use. Preferably, R¹¹ is CO₂R¹⁴; R¹² is hydrogen; R¹³ and R¹⁴ are, independently, 3′-phthalidyl, hydrogen, —CHR¹⁵O₂CXR¹⁶, or R¹⁵ is hydrogen or methyl; R¹⁶ is (1-10C) alkyl or aryl; and X is 0 or a bond. Representative compounds from this preferred group of formula I compounds include (1S,2S,5R,6S)-2-amino-bicyclo[3.1.0]hexane-2,6-dicarboxylic acid 2,6-dibenzoyloxymethyl ester hydrochloride, (1S,2S,5R,6S)-2-amino-bicyclo[3.1.0]hexane-2,6-dicarboxylic acid 2,6-dicyclohexyloxycarbonyloxymethyl ester, (1S,2S,5R,6S)-2-amino-bicyclo[3.1.0]hexane-2,6-dicarboxylic acid 2,6-di(1′-cyclohexyloxycarbonyloxy-ethyl) ester hydrochloride, (1S,2S,5R,6S)-2-amino-bicyclo[3.1.0]×hexane-2,6-dicarboxylic acid 2,6-di-(3′-phthalidyl) ester hydrochloride, (1S,2S,5R,6S)-2-amino-bicyclo[3.10]hexane-2,6-dicarboxylic acid 2-isopropyloxycarbonyloxymethyl ester hydrochloride, and (1S,2S,5R,6S)-2-amino-bicyclo[3.1.0]hexane-2,6-dicarboxylic acid 6-tert-butylcarbonyloxymethyl ester.

Certain compounds of the present invention are more preferred for use in providing prodrug forms. More preferably, R¹⁵ is hydrogen and X is a bond. Representative compounds from this more preferred group of compounds include (1S,2S,5R,6S)-2-amino-bicyclo[3.1.0]hexane-2,6-dipropanoyloxymethyl ester hydrochloride, (1S,2S,5R,6S)-2-amino-bicyclo[3.1.0]hexane-2,6-dicarboxylic acid 2-(4′-methoxybenzoyloxy)methyl ester hydrochloride, and (1S,2S,5R,6S)-2-amino-bicyclo[3.1.0]hexane-2,6-dicarboxylic acid 2-(naphthalene-1′-carbonyloxymethyl) ester hydrochloride.

Most preferably, the compound of formula (I) is (1S,2S,5R,6S)-2-amino-bicyclo[3.1.0]hexane-2,6-dicarboxylic acid 2,6-dipivaloyloxymethyl ester hydrochloride or (1S,2S,5R,6S)-bis-(5-methyl-2-oxo-[1,3]dioxolen-4-ylmethyl) 2-amino-bicyclo[3.1.0]hexane-2,6-dicarboxylate ethanesulfonic acid or a pharmaceutically acceptable salt thereof.

While all the compounds of formula II of the present invention are believed to be useful for the synthesis of compounds of formula I, certain compounds are preferred. Preferably, R^{m} is tert-butoxycarbonyl and R¹³ and R¹⁴ are H; or R^{m} is allyloxycarbonyl, R¹³ is hydrogen and R¹⁴ is (2-4C) alkenyl group, for example an allyl group.

Also useful for the synthesis of compounds of formula I are compounds where the C-2 amino and carboxy groups are protected in the form of a cyclic ring. Preferably, the cyclic ring is an oxazolidinone that is spiro fused to the 2-postion of bicyclo[3.1.0]hexane-6-carboxylic acid, for example a compound of formula IV.

The compounds of formula II of the present invention are generally synthesized from compounds of formula III where R¹³ and R¹⁴ are both hydrogen. The compounds of formula III are prepared as described in U.S. Pat. No. 5,750,566 which is incorporated by reference in its entirety.

Generally, compounds of formula II in which R¹³ is —CHR¹⁵O₂CXR¹⁶ may be prepared by directly reacting compounds of formula II where R¹³ is hydrogen and R¹⁴ is a carboxy protecting group. Compounds of formula II in which R¹³ and R¹⁴ are —CHR¹⁵O₂CXR¹⁶ may be prepared by reacting compounds of formula II where R¹³ and R¹⁴ are both hydrogen.

More specifically, compounds of formula III are reacted with amine protecting agents such as allyl chloroformate in the presence of a suitable aqueous base such as sodium bicarbonate in a suitable solvent such as dioxane to produce compounds of formula II. Compounds of formula II are then reacted with carboxy protecting agents such as allyl alcohol, EDCI and HOBt in the presence of a suitable base such as triethylamine in a convenient solvent such as dichloromethane to provide compounds of formula V as shown in scheme 2.

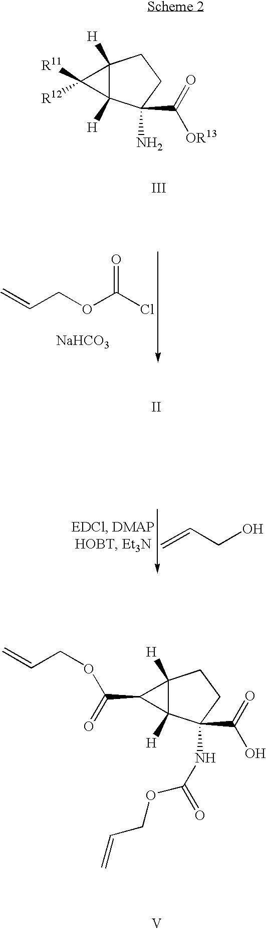

Compounds of formula VI may be prepared from compounds of formula V, a carboxlic acid, with a substituted alkyl halide of formula QCHR¹⁵O₂CXR¹⁶ in which Q is, for example, chlorine or bromine as shown in scheme 3. The reaction is conveniently performed in the presence of a base, such as potassium carbonate and an activating agent, such as sodium iodide. Convenient solvents include dimethylformamide.

Compounds of formula VI are reacted with a metal catalyst such as tetrakistriphenylphosphine palladium(0) to produce compounds of formula I where R¹¹ is CO₂R¹⁴, R¹² and R¹⁴ are hydrogen, and R¹³ is —CHR¹⁵O₂CXR¹⁶. The reaction is performed in the presence of a metal catalyst regenerating agent such as 1,3-dimethylbarbituric acid in a convenient solvent such as dichloromethane. The acid addition salts may be prepared by the reaction of an acid such as hydrogen chloride gas with a compound of formula I. Convenient solvents include ethyl acetate.

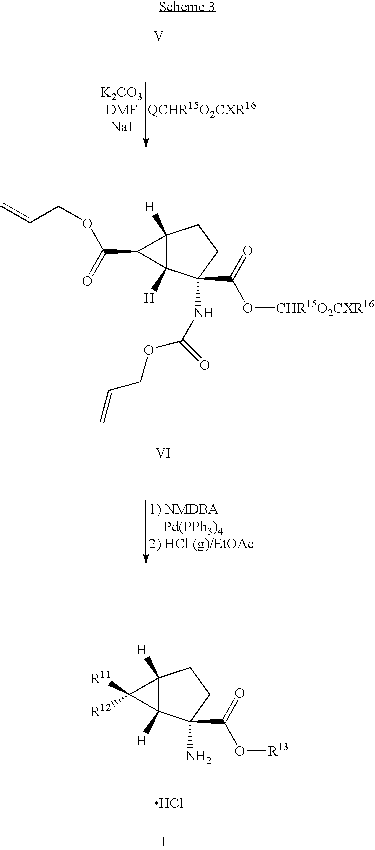

Generally, compounds of formula II in which R¹³ and R¹⁴ are both —CHR¹⁵O₂CXR¹⁶ may be prepared by reacting compounds of formula II where R¹³ and R¹⁴ are both hydrogen as shown in scheme 4. More specially, compounds of formula II may be prepared by reacting compounds of formula III with amine protecting agents such as di-tert-butyl dicarbonate in the presence of a suitable aqueous base such as sodium hydroxide in a suitable solvent such as dioxane to produce compounds of formula II where Rₘ is tert-butyloxycarbonyl.

Compounds of formula VII may be prepared from compounds of formula II, a dicarboxlic acid, with a substituted alkyl halide of formula QCHR¹⁵O₂CXR¹⁶ in which Q is a suitable leaving group such as chlorine. The reaction is conveniently performed in the presence of a base, such as potassium carbonate and an activating agent, such as sodium iodide. Convenient solvents include dimethylformamide.

Compounds of formula VII are reacted with amine deprotecting agents such as an acid to provide compounds of formula I in which R¹¹ is CO₂R¹⁴, R¹³ and R¹⁴ are both —CHR¹⁵O₂CXR¹⁶ or 5-methyl-2-oxo-[1,3]dioxolen-4-ylmethyl and R¹² is hydrogen. Convenient acids include trifluoroacetic acid or hydrogen chloride gas saturated in a convenient solvent such as ethyl acetate. Such conditions may produce the corresponding acid salt of the compound of formula I as an amorphous or, directly, a crystalline solid. In the case of an amorphous solid, subsequent crystallization may occur from suitable solvents. In the case of a crystalline solid formed under reaction conditions, filtration of the reaction mixture may afford the crystalline salt directly. The zwitterionic compound of formula I may be prepared by treatment of the acid salt of formula I with a suitable reagent such as a base, for example aqueous sodium bicarbonate, or propylene oxide. Additional acid salts may be prepared by treating the zwitterionic compound of formula I with a suitable acid. Suitable acids include ethanesulfonic acid in a convenient solvent such as methylene chloride.

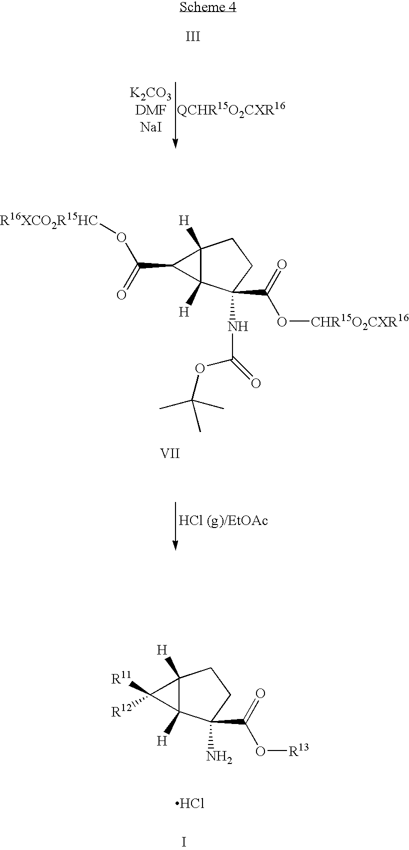

Generally, compounds of formula I in which R¹³ is hydrogen and R¹⁴ is —CHR¹⁵O₂CXR¹⁶ may be prepared by reacting compounds of formula IV where R¹⁴ is hydrogen as shown in scheme 5. More specially, compounds of formula IV may be prepared by reacting compounds of formula II where Rₘ is an amine protecting group such as allyloxycarbonyl with an aldehyde such as paraformaldehyde in the presence of a suitable acid catalyst such as p-toluene sulphonic acid. The reaction may be carried out in a suitable solvent such as benzene with convenient removal of water such as azeotropic distillation.

Compounds of formula IIX may be prepared from compounds of formula IV where R¹⁴ is hydrogen with a substituted alkyl halide of formula QCHR¹⁵O₂CXR¹⁶ in which Q is a suitable leaving group such as chlorine. The reaction is conveniently performed in the presence of a base, such as potassium carbonate. An activating agent, such as sodium iodide may be added to facilitate the reaction. Convenient solvents include dimethylformamide.

Compounds of formula IIX are reacted with a metal catalyst such as tetrakistriphenylphosphine palladium(0) to produce compounds of formula I where R¹¹ is CO₂R¹⁴, R¹² and R¹³ are hydrogen, and R¹⁴ is —CHR¹⁵O₂CXR¹⁶. The reaction is conveniently performed in the presence of a metal catalyst regenerating agent such as 1,3-dimethylbarbituric acid in a suitable solvent such as dichloromethane. The acid addition salts may be prepared by the reaction of an acid such as hydrogen chloride gas with a compound of formula I. Convenient solvents include ethyl acetate. The zwitterionic compound of formula I may be prepared by treatment of the acid salt of formula I with a suitable acid scavenger such as propylene oxide in a suitable solvent such as methanol.

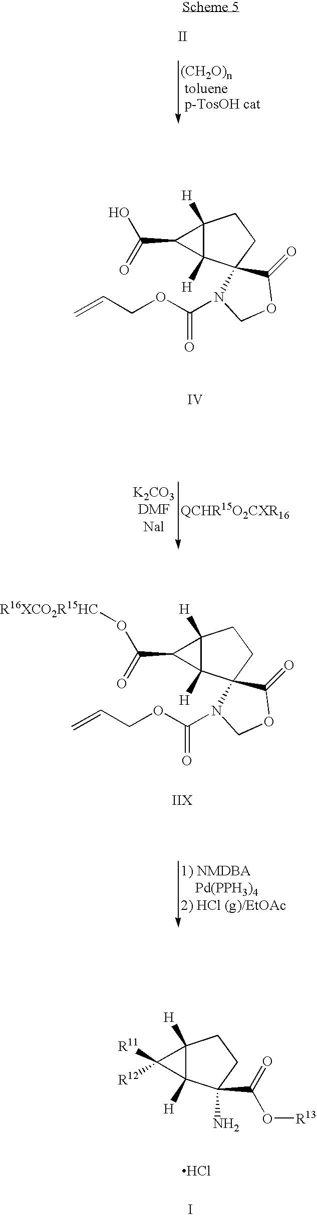

The term “affecting” refers to a formula I compound acting as an agonist at an excitatory amino acid receptor. The term “excitatory amino acid receptor” refers to a metabotropic glutamate receptor, a receptor that is coupled to cellular effectors via GTP-binding proteins. The term “cAMP-linked metabotropic glutamate receptor” refers to a metabotropic receptor that is coupled to inhibition of adenylate cyclase activity.

The term “neurological disorder” refers to both acute and chronic neurodegenerative conditions, including cerebral deficits subsequent to cardiac bypass surgery and grafting, cerebral ischemia (for example stroke resulting from cardiac arrest), spinal cord trauma, head trauma, Alzheimer's Disease, Huntington's Chorea, amyotrophic lateral sclerosis, AIDS-induced dementia, perinatal hypoxia, hypoglycemic neuronal damage, ocular damage and retinopathy, cognitive disorders, idiopathic and drug-induced Parkinson's Disease. This term also includes other neurological conditions that are caused by glutamate dysfunction, including muscular spasms, migraine headaches, urinary incontinence, drug tolerance, withdrawal, and cessation (i.e. opiates, benzodiazepines, nicotine, cocaine, or ethanol), smoking cessation, emesis, brain edema, chronic pain, sleep disorders, convulsions, Tourette's syndrome, attention deficit disorder, and tardive dyskinesia.

The term “psychiatric disorder” refers to both acute and chronic psychiatric conditions, including schizophrenia, anxiety and related disorders (e.g. panic attack and stress-related cardiovascular disorders), depression, bipolar disorders, psychosis, and obsessive compulsive disorders.

If not commercially available, the necessary starting materials for the above procedures may be made by procedures which are selected from standard techniques of organic and heterocyclic chemistry, techniques which analogous to the syntheses of known, structurally similar compounds, and the procedures described in the Examples, including novel procedures.

As used herein the term “effective amount” refers to the amount or dose of the compound, upon single or multiple dose administration to the patient, which provides the desired effect in the patient under diagnosis or treatment.

An effective amount can be readily determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the use of concomitant medication; and other relevant circumstances. For example, a typical daily dose may contain from about 25 mg to about 300 mg of the active ingredient. The compounds can be administered by a variety of routes including oral, rectal, transdermal, subcutaneous, intravenous, intramuscular, bucal or intranasal routes. Alternatively, the compound may be administered by continuous infusion.

As used herein the term “patient” refers to a mammal, such as a mouse, guinea pig, rat, dog or human. It is understood that the preferred patient is a human.

The term “treating” (or “treat”) as used herein includes its generally accepted meaning which encompasses prohibiting, preventing, restraining, and slowing, stopping, or reversing progression of a resultant symptom. As such, the methods of this invention encompass both therapeutic and prophylactic administration.

The ability of compounds to modulate metabotropic glutamate receptor function may be demonstrated by examining their ability to influence either cAMP production (mGluR 2, 3, 4, 6, 7 or 8) or phosphoinositide hydrolysis (mGluR 1 or 5) in cells expressing these individual human metabotropic glutamate receptor (mGluR) subtypes. (D. D. Schoepp, et al., Neuropharmacol., 1996, 35, 1661-1672 and 1997, 36, 1-11).

The abililty of formula I compounds to treat anxiety or a related disorder may be demonstrated using the well known fear potentiated startle and elevated plus maze models of anxiety described respectively in Davis, Psychopharmacology, 62:1; 1979 and Lister, Psychopharmacol, 92: 180-185; 1987.

### In Vivo Exposure as Measured by Rat Plasma Concentration

To study the in vivo exposure of LY354740 following oral dosing of compounds of the present invention in comparison to LY354740, studies measuring the plasma concentrations of LY354740 in rats were performed.

Mature Fischer 344 male rats (190-270 gram) were obtained from Harlan Sprague-Dawley, Cumberland, Ind., USA and acclimated in the study housing for 3 days. On day 4, test compounds were dissolved in buffered water (1 mg/ml=test compound/20 mM potassium dihydrogen phosphate, pH=2) and given orally as a single 5 mg/kg dose. Blood samples were collected through orbital sinus or cardiac puncture (last time point) at 0.5 and 1 hour or, alternatively, 1 and 3 hours. Plasma samples were stored at −20° C. in the presence of phenylmethylsulfonyl fluoride, a protease inhibitor, prior to analysis. Plasma samples and internal standard compounds were pretreated by solid phase extraction (SAX support, methanol/water/dilute acetic acid). As shown in the second row of Table 1, below, the plasma concentrations (ng/ml) of LY354740 for each test compound were determined by LC/MS/MS and are presented as a sum of the concentrations at the 0.5 and 1 hour or, alternatively, 1 and 3 hour sample time points.

TABLE 1
Comparison of plasma concentrations of LY354740 and
compounds of the present invention

| | Plasma Concentration of |
|---|---|
| Compound | LY354740, ng/ml |
| (@ 5 mg/kg p. o.) | (sum of 0.5 and 1 hour) |
| LY354740 | 466 |
| Example 14 | 1494 |
| Example 15 | 3600 |
| Example 17 | 2403 |
| Example 21 | 1545 |
| Example 42 | 3898 |
| Example 43 | 1831 |
| Example 47 | 3225 |
| Example 50 | 2197 |
| Example 53 | 1450 |
| Example 54 | 1354 |
| Example 57 | 4786 |

The compounds of the present invention are preferably formulated prior to administration. Therefore, another aspect of the present invention is a pharmaceutical formulation comprising a compound of formula I a pharmaceutically acceptable metabolically labile ester thereof, or a pharmaceutically acceptable salt thereof, and a pharmaceutically-acceptable carrier, diluent, or excipient. The present pharmaceutical formulations are prepared by known procedures using well-known and readily available ingredients. In making the compositions of the present invention, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material which acts as a vehicle, excipient, or medium for the active ingredient. The compositions can be in the form of tablets, pills, powders, lozenges, sachets, cachets, elixirs, suspensions, emulsions, solutions, syrups, aerosols, ointments containing, for example, up to 10% by weight of active compound, soft and hard gelatin capsules, suppositories, sterile injectable solutions, and sterile packaged powders.

Some examples of suitable carriers, excipients, and diluents include lactose, dextrose, sucrose, sorbitol, mannitol, starches, gum, acacia, calcium phosphate, alginates, tragacanth, gelatin, calcium silicate, microcrystalline cellulose, polyvinylpyrrolidone, cellulose, water syrup, methyl cellulose, methyl and propyl hydroxybenzoates, talc, magnesium stearate, and mineral oil. The formulations can additionally include lubricating agents, wetting agents, emulsifying and suspending agents, preserving agents, sweetening agents, or flavoring agents. Compositions of the invention may be formulated so as to provide quick, sustained, or delayed release of the active ingredient after administration to the patient by employing procedures well known in the art.

The compositions are preferably formulated in a unit dosage form, each dosage containing from about 5 mg to about 500 mg, more preferably about 25 mg to about 300 mg of the active ingredient. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical carrier, diluent, or excipient.

The following Examples further illustrate the compounds of the present invention and the methods for their synthesis. The Examples are not intended to be limiting to the scope of the invention in any respect, and should not be so construed. All experiments were run under a positive pressure of dry nitrogen or argon. All solvents and reagents were purchased from commercial sources and used as received, unless otherwise indicated. Dry tetrahydrofuran (THF) was obtained by distillation from sodium or sodium benzophenone ketyl prior to use. Proton nuclear magnetic resonance (¹H NMR) spectra were obtained on a GE QE-300 spectrometer at 300.15 MHz, a Bruker AM-500 spectrometer at 500 MHz, a Bruker AC-200P spectrometer at 200 MHz or a Varian Inova at 500 MHz. Free atom bombardment mass spectroscopy (FABMS) was performed on a VG ZAB-2SE instrument. Field desorption mass spectroscopy (FDMS) was performed using either a VG 70SE or a Varian MAT 731 instrument. Optical rotations were measured with a Perkin-Elmer 241 polarimeter. Chromatographic separation on a Waters Prep 500 LC was generally carried out using a linear gradient of the solvents indicated in the text. The reactions were generally monitored for completion using thin layer chromatography (TLC). Thin layer chromatography was performed using E. Merck Kieselgel 60 F₂₅₄ plates, 5 cm×10 cm, 0.25 mm thickness. Spots were detected using a combination of UV and chemical detection (plates dipped in a ceric ammonium molybdate solution [75 g of ammonium molybdate and 4 g of cerium (IV) sulfate in 500 mL of 10% aqueous sulfuric acid] and then heated on a hot plate). Flash or silica gel chromatography was performed as described by Still, et al. Still, Kahn, and Mitra, J. Org. Chem., 43, 2923 (1978). Elemental analyses for carbon, hydrogen, and nitrogen were determined on a Control Equipment Corporation 440 Elemental Analyzer, or were performed by the Universidad Complutense Analytical Centre (Facultad de Farmacia, Madrid, Spain). Melting points were determined in open glass capillaries on a Gallenkamp hot air bath melting point apparatus or a Büchi melting point apparatus, and are uncorrected. The number in parenthesis after the compound name refers to the compound number.

The abbreviations, symbols and terms used in the examples have the following meanings.

Ac=acetyl

Anal.=elemental analysis

Bn or Bzl=benzyl

Bu=butyl

BOC=tert-butoxycarbonyl

calcd=calculated

D₂O=deuterium oxide

DCC=dicyclohexylcarbodiimide

DIBAL-H=diisobutyl aluminum hydride

DMAP=dimethylaminopyridine

DMF=dimethylformamide

DMSO=dimethylsulfoxide

EDCI=N-ethyl-N′N′-dimethylaminopropyl carbodiimide

Et=ethyl

EtOAc=ethyl acetate

EtOH=ethanol

FAB=Fast Atom Bombardment (Mass Spectrascopy)

FDMS=field desorption mass spectrum

HOAt=1-hydroxy-7-azabenzotriazole

HOBt=1-hydroxybenzotriazole

HPLC=High Performance Liquid Chromatography

HRMS=high resolution mass spectrum

i-PrOH=isopropanol

IR=Infrared Spectrum

L=liter

Me=methyl

MeOH=methanol

MPLC=Medium Pressure Liquid Chromatography

Mp=melting point

MTBE=t-butyl methyl ether

NBS=N-bromosuccinimide

NMDBA=1,3-dimethylbarbituric acid

NMR=Nuclear Magnetic Resonance

Ph=phenyl

p.o.=oral administration

i-Pr=isopropyl

Rochelle's Salt=potassium sodium tartrate

SM=starting material

TBS=tert-butyldimethylsilyl

TEA=triethylamine

Temp.=temperature

TFA=trifluoroacetic acid

THF=tetrahydrofuran

TLC=thin layer chromatography

t-BOC=tert-butoxycarbonyl

### Example Preparation 1

Synthesis of (1S,2S,5R,6S)-2-allyloxycarbonylamino-bicyclo[3.1.0]hexane-2,6-dicarboxylic acid 6-allyl ester

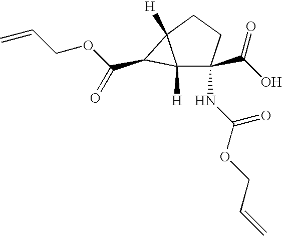

A. (1S,2S,5R,6S)-2-Allyloxycarbonylamino-bicyclo[3.1.0]hexane-2,6-dicarboxylic acid

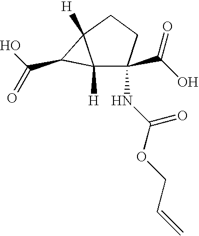

(1S,2S,5R,6S)-2-Amino-bicyclo[3.1.0]hexane-2,6-dicarboxylic acid (15.0 g, 73.9 mmol) was slowly dissolved in 250 mL of NaHCO₃ sat. (250 mL). After complete solution, dioxane (100 mL) and allyl chloroformate (15.7 mL, 147.8 mmol) were added at room temperature and the mixture was stirred overnight. The reaction mixture was diluted with water (100 mL) and washed with ethyl acetate (3×). The organic layer was extracted once with sat. NaHCO₃. The combined aqueous layers were acidified to pH 1 with 4N HCl and extracted with ethyl acetate (2×). The organic layer was dried over magnesium sulfate, filtered and concentrated to provide an oil (13.4 g, 67% yield) that was used without further purification.

¹H-NMR (CD₃OD) δ: 6.01-5.82 (m, 1H); 5.35-5.13 (m, 2H); 4.51 (d, J=5.1 Hz, 2H); 2.48-1.78 (m, 5H); 1.69-1.62 (m, 1H); 1.45-1.29 (m, 1H). ¹³C-NMR (CD₃OD) δ: 176.7, 176.6, 158.3, 134.2, 117.4, 67.3, 66.3, 35.8, 33.1, 29.9, 27.0, 22.0.

B. (1S,2S,5R,6S)-2-Allyloxycarbonylamino-bicyclo[3.1.0]hexane-2,6-dicarboxylic acid 6-allyl ester

To a suspension of (1S,2S,5R,6S)-2-allyloxycarbonylamino-bicyclo[3.1.0]hexane-2,6-dicarboxylic acid (13.4 g, 49.8 mmol) in dichloromethane (400 mL), N-ethyl-N′-dimethylaminopropylcarbodiimide (9.55 g, 49.8 mmol) and dimethylaminopyridine (0.61 g, 5.0 mmol) were added at room temperature under nitrogen. Allyl alcohol (3.4 mL, 49.8 mmol) was added and the mixture was stirred overnight at room temperature. The reaction mixture was diluted with dichloromethane and washed with water (2×). The organic layer was dried over magnesium sulfate, filtered and concentrated to provide the title compound (6.8 g, 44% yield) as an oil.

¹H-NMR (CD₃OD) δ: 6.01-5.82 (m, 1H); 5.35-5.13 (m, 2H); 4.51 (d, J=5.1 Hz, 2H); 2.48-1.78 (m, 5H); 1.69-1.62 (m, 1H); 1.45-1.29 (m, 1H).

### Example Preparation 2

Synthesis of (1S,2S,5R,6S)-2-tert-Butoxycarbonylamino-bicyclo[3.1.0]hexane-2,6-dicarboxylic acid

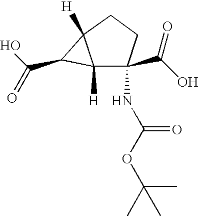

A 1 L flask was charged with (1S,2S,5R,6S)-2-amino-bicyclo[3.1.0]hexane-2,6-dicarboxylic acid monohydrate (24.4 g, 0.12 mol, 1 equiv), dioxane (200 mL) and di-tert-butyl dicarbonate (52.4 g, 0.24 mol, 2.0 equiv). The suspension was vigorously stirred while sodium hydroxide 1N (420 mL, 3.5 equiv) was added. The mixture was stirred for 2 days, then 2.0 more equiv of di-tert-butyl dicarbonate were added and the reaction stirred for 3 additional days at rt. After 5 total days of reaction, water (400 mL) was added to dissolve the salts. The aqueous layer was extracted with ethyl acetate (4×100 mL) to remove the excess of reagent, and then taken to ca. pH=2 using 6 N hydrochloric acid. The acidic aqueous phase was then extracted using ethyl ether (6×200 mL). The combined organic layers were washed with water (250 mL) and brine (250 mL). After drying over sodium sulfate, solvents were evaporated under vacuum to afford a foamy white solid (26.4 g). 77% Yield. mp 100-101° C.

[α]_{D}²⁵=−41.1° (c=1.0, MeOH).

¹H NMR (Methanol-d₄) δ: 4.98 (brs, 1H), 2.44 (dd, 1H, J=6.2, 2.6 Hz), 2.19-1.92 (m, 4H), 1.62 (t, 1H, J=2.8 Hz), 1.43 (s, 9H), 1.29 (m, 1H).

³C NMR (Methanol-d₄) δ: 175.6, 175.2, 158.2, 60.1, 34.6, 31.9, 28.4, 27.2, 25.6, 20.6.

MS (Neg. Electrospray): 284.2 (M⁺−H).

### Example Preparation 3

Synthesis of (1′S,2′S,5′R,6′S)-3-Allyloxycarbonyl-5-oxo-oxazolidinone-4-spiro-2′-bicyclo[3.1.0]hexane-6′-carboxylic acid

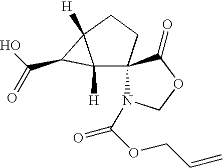

(1S,2S,5R,6S)-2-Allyloxycarbonylamino-bicyclo[3.1.0]hexane-2,6-dicarboxylic acid, Example A.1, (19.17 g, 64.33 mmol), paraformaldehyde (7.73 g, 257.32 mmol) and p-toluenesulphonic acid (0.612 g, 3.216 mmol) were slurried in 200 mL of benzene and refluxed with azeotropic removal of water for two hours. The mixture was cooled to room temperature and diluted with 200 mL of ethyl acetate, washed twice with brine and dried over MgSO₄. Concentration afforded a slighly hygroscopic solid (17.6 g, 97%) >95% pure by NMR.

¹H-NMR (CDCl₃, 200.15 MHz): 6.10-5.90 (m, 1H); 5.41-5.21 (m, 4H); 4.65 (dt, J=5.6, 1.3 Hz, 2H); 2.51-2.49 (m, 1H); 2.33-2.18 (m, 2H); 2.04-1.92 (m, 3H); 1.75-1.70 (m, 1H). ¹³C-NMR (CD₃OD, 50 MHz): 176.1, 175.4, 153.0, 133.5, 118.9, 78.4, 67.8, 67.4, 33.1, 27.2 (×2), 26.0, 23.9.

### General Procedure 1

General Procedure for Esterification of (1S,2S,5R,6S)-2-Allyloxycarbonylamino-bicyclo[3.1.0]hexane-2,6-dicarboxylic acid 6-allyl ester or (1′S,2′S,5′R,6′S)-3-Allyloxycarbonyl-5-oxo-oxazolidinone-4-spiro-2′-bicyclo[3.1.0]hexane-6′-carboxylic acid

(1S,2S,5R,6S)-2-Allyloxycarbonylamino-bicyclo[3.1.0]hexane-2,6-dicarboxylic acid 6-allyl ester (Example Preparation 1) (1.0 equiv) or (1′S,2′S,5′R,6′S)-3-allyloxycarbonyl-5-oxo-oxazolidinone-4-spiro-2′-bicyclo[3.1.0]hexane-6′-carboxylic acid (Example Preparation 3) (1.0 equiv) was dissolved in dry dimethylformamide (0.5 M solution) under nitrogen and potassium carbonate or cesium carbonate (1.0 equiv) was added at room temperature or 0° C. After 5 min, the corresponding halide (1.0 equiv) and sodium iodide (1.0 equiv) were added. The reaction mixture was stirred at room temperature overnight. Water was added and the aqueous phase was extracted with two portions of ethyl acetate. The combined organic phase was washed with cold water, dried over magnesium sulfate and concentrated to dryness. The crude mixture was purified by silica gel flash-chromatography using the appropriate eluent (typically mixtures of hexanes/ethyl acetate).

### EXAMPLE 1

(1S,2S,5R,6S)-2-Allyloxycarbonylamino-bicyclo[3.1.0]hexane-2,6-dicarboxylic acid 6-allyl ester 2-(3′-phthalidyl) ester

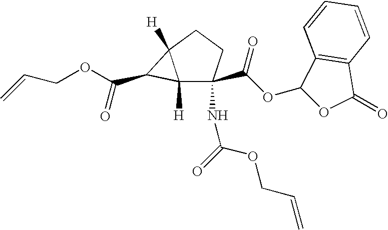

The title compound was prepared according to General Procedure 1 using 3-bromophthalide (which was obtained from phthalide as described by Koten et al. Org. Synth. Coll. Vol. V, 145-147).

92% Yield. Foamy white solid. Mixture of diastereomers.

¹H NMR (CDCl₃) δ (Mixture of diastereomers): 7.94-7.91 (m, 1H),7.78-7.56 (m, 3H), 7.42 (brs, 1H), 5.96-5.78 (m, 2H), 5.60 (brs, 1H), 5.34-5.15 (m, 4H), 4.56-4.51 (m, 4H), 2.61-2.54 (m, 1H), 2.44-2.34 (m, 1H), 2.22-1.94 (m, 3H), 1.79-1.72 (m, 1H), 1.39-1.21 (m, 1H).

¹³C NMR (CDCl₃) δ (Mixture of diastereomers): 171.6, 171.4, 171.1, 167.6, 155.5, 144.1, 134.8, 132.2, 131.9, 131.8, 131.2, 126.3, 125.6, 123.7, 118.5, 118.4, 117.9, 93.3, 66.7, 66.6, 65.8, 65.5, 65.4, 34.0, 32.3, 28.4, 28.0, 26.7, 26.0, 21.2, 21.0.

MS (Electrospray): 464.2 (M⁺+Na).

### EXAMPLE 2

(1S,2S,5R,6S)-2-Allyloxycarbonylamino-bicyclo [3.1.0]hexane-2,6-dicarboxylic acid 6-allyl ester 2-isopropyloxycarbonyloxymethyl ester

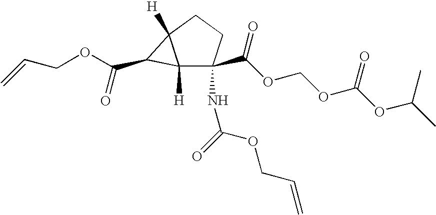

The title compound was prepared according to General Procedure 1 using chloromethyl isopropyl carbonate (which was obtained from chloromethyl chloroformate as described by Patonay, T. et al, Synth. Commun. 1990, 20, 2865-2885 and Davidsen et al. J. Med. Chem. 1994, 37, 4423-4429).

69% Yield. Colorless syrup.

[α]_{D}²⁵=−6.36° (c=0.66, CHCl₃).

¹H NMR (CDCl₃) δ: 5.92-5.77 (m, 2H), 5.89 (brs, 1H), 5.80 (brs, 2H), 5.35-5.17 (m, 4H), 4.91 (m, 1H), 4.54 (m, 4H), 2.45-2.37 (m, 2H), 2.10-1.95 (m, 3H), 1.78 (t, 1H, J=2.6 Hz), 1.33-1.26 (m, 1H), 1.31 (d, 6H, J=6.4 Hz).

¹³C NMR (CDCl₃) δ: 171.6, 171.1, 155.4, 153.0, 132.3, 131.8, 118.1; 117.5, 82.4, 72.8, 66.3, 65.5, 65.1, 34.1, 32.0, 28.2, 26.3, 21.4, 21.3, 20.9.

MS (Electrospray): 425.8 (M⁺+Na).

### EXAMPLE 3

(1S,2S,5R,6S)-2-Allyloxycarbonylamino-bicyclo[3.1.0]hexane-2,6-dicarboxylic acid 6-allyl ester 2-(4′-methoxy-benzoyloxymethyl) ester

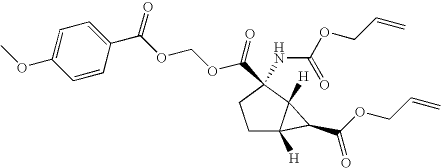

The title compound was prepared according to General Procedure 1 using chloromethyl p-methoxybenzoate (which was readily obtained as described by Davidsen et al. J. Med. Chem. 1994, 37, 4423-4429).

52% Yield.

¹H-NMR (CDCl₃): 7.99 (d, J=9.1 Hz, 2H), 6.90 (d, J=9.1 Hz, 2H), 6.00 (s, 2H), 5.93-5.70 (m, 2H), 5.56 (s, 1H), 5.31-5.09 (m, 4H), 4.51 (d, J=5.6 Hz, 2H), 4.42 (d, J=5.6 Hz, 2H), 3.83 (s, 3H), 2.49-2.38 (m, 2H), 2.16-1.71 (m, 4H), 1.34-1.18 (m, 1H).

### EXAMPLE 4

(1S,2S,5R,6S)-2-Allyloxycarbonylamino-bicyclo[3.1.0]hexane-2,6-dicarboxylic acid 6-allyl ester 2-(naphthalene-2′-carbonyloxymethyl) ester

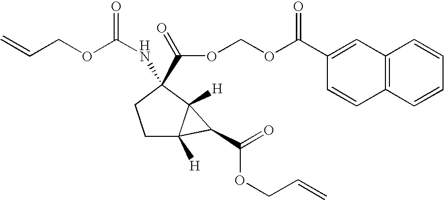

The title compound was prepared according to General Procedure 1 using chloromethyl 2-napthtoate (which was obtained using a similar procedure to the one described by Davidsen et al. J. Med. Chem. 1994, 37, 4423-4429).

63% yield. Oil.

IR (KBr) ν: 3360, 3059, 2988, 2939, 2876, 1732, 1653, 1508, 1458, 1417, 1329, 1283, 1258, 1178, 1156, 981 cm⁻¹.

¹H NMR (CDCl₃) δ: 8.66 (m, 1H), 8.07 (m, 1H), 7.95 (m, 1H), 7.88 (m, 2H), 7.66-7.51 (m, 2H), 6.13 (s, 2H), 5.98-5.72 (m, 2H), 5.48 (brs, 1H), 5.18 (m, 4H), 4.54 (m, 2H), 4.44 (m, 2H), 2.56-2.46 (m, 2H), 2.27-1.91 (m, 3H), 1.76 (t, 1H, J=3.0 Hz), 1.30 (m, 1H).

¹³C NMR (CDCl₃) δ: 171.6, 171.5, 165.3, 155.5, 135.9, 131.9, 129.5, 128.6, 128.3, 127.8, 126.8, 126.0, 125.2, 132.4, 132.3, 118.5, 117.8, 80.4, 66.7, 65.8, 65.4, 34.3, 32.4, 28.3, 26.6, 21.2.

MS (Electrospray): 516.1 (M⁺+Na), 494.1 (M⁺+H).

### EXAMPLE 5

(1S,2S,5R,6S)-2-Allyloxycarbonylamino-bicyclo[3.1.0]hexane-2,6-dicarboxylic acid 6-allyl ester 2-(naphthalene-1′-carbonyloxymethyl) ester

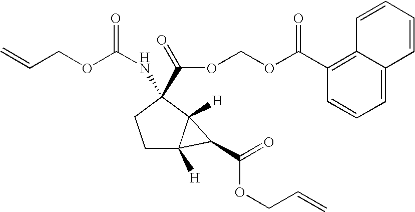

The title compound was prepared according to General Procedure 1 using chloromethyl 1-napthtoate (which was obtained using a similar procedure to the one described by Davidsen et al. J. Med. Chem. 1994, 37, 4423-4429).

78% yield. Oil.

IR (KBr) ν: 3366, 3080, 3051, 2991, 2945, 2874, 1732, 1630, 1508, 1458, 1417, 1329, 1280, 1248, 1179, 1159, 1119, 987 cm⁻¹.

1H NMR (CDCl₃) δ: 8.95 (m, 1H), 8.29 (m, 1H), 8.06 (m, 1H), 7.89 (m, 1H), 7.68-7.47 (m, 3H), 6.15 (s, 2H), 5.99-5.69 (m, 2H), 5.51 (brs, 1H), 5.20 (m, 4H), 4.54 (m, 2H), 4.43 (m, 2H), 2.57-2.45 (m, 2H), 2.28-1.92 (m, 3H), 1.77 (t, 1H, J=3.0 Hz), 1.30 (m, 1H).

¹³C NMR (CDCl₃) δ: 171.6, 171.5, 165.6, 155.5, 134.3, 133.8, 131.5, 131.4, 128.6, 128.1, 126.3, 125.6, 125.2, 124.5, 132.3, 131.9, 118.5, 117.8, 80.3, 66.7, 65.8, 65.4, 34.4, 32.4, 28.3, 26.6, 21.2.

MS (Electrospray): 516.1 (M⁺+Na), 494.1 (M⁺+H).

### EXAMPLE 6

(1S,2S,5R,6S)-2-Allyloxycarbonylamino-bicyclo[3.1.0]hexane-2,6-dicarboxylic acid 6-allyl ester 2-cyclohexyloxycarbonyloxymethyl ester

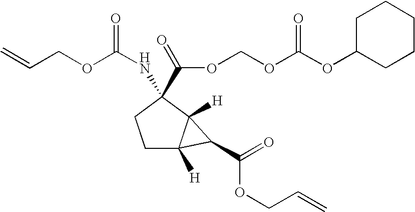

The title compound was prepared according to General Procedure 1 using chloromethyl cyclohexyl carbonate (which was obtained from chloromethyl chloroformate as described by Patonay, T. et al, Synth. Commun. 1990, 20, 2865-2885 and Davidsen et al. J. Med. Chem. 1994, 37, 4423-4429).

65% yield. Oil.

IR (KBr) ν: 3358, 2941, 2863, 1761, 1728, 1518, 1452, 1415, 1323, 1278, 1261, 1155 cm⁻¹.

¹H NMR (CDCl₃) δ: 5.88 (m, 2H), 5.80 (s, 2H), 5.45 (brs, 1H), 5.25 (m, 4H), 4.66 (m, 1H), 4.53 (m, 4H), 2.53-2.40 (m, 2H), 2.24-1.88 (m, 5H), 1.81-1.70 (m, 3H), 1.60-1.20 (m, 7H).

¹³C NMR (CDCl₃) δ: 171.6, 171.2, 155.5, 153.2, 132.4, 132.0, 118.5, 117.9, 82.6, 77.8, 66.6, 65.9, 65.5, 34.4, 32.4, 31.3, 28.3, 26.6, 25.1, 23.5, 21.2.

MS (Electrospray): 488.1 (M⁺+Na), 466.2 (M⁺+H).

### EXAMPLE 7

Synthesis of (1′S,2′S,5′R,6′S)-3-Allyloxycarbonyl-5-oxo-oxazolidinone-4-spiro-2′-bicyclo[3.1.0]hexane-6′-carboxylic acid 6′-tert-butylcarbonyloxymethyl ester

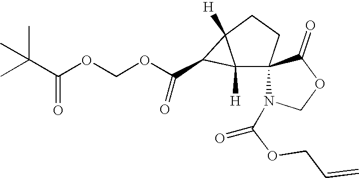

The title compound was prepared according to General Procedure 1 using chloromethyl pivalate.

67% Yield. Colorless syrup.

[α]_{D}²⁵=+47.0° (c=1.52, CHCl₃).

¹H NMR (CDCl₃) δ: 5.94 (m, 1H), 5.72 (AB system, 2H), 5.38-5.25 (m, 2H), 5.34 (brs, 2H), 4.64 (brd, 2H, J=5.9 Hz), 2.58 (brs, 1H), 2.31 (m, 2H), 2.05-1.98 (m, 3H), 1.74 (m, 1H), 1.21 (s, 9H),

¹³C NMR (CDCl₃) δ: 176.5, 173.0, 170.7, 151.1, 131.6, 118.7, 79.3, 76.7, 66.1, 66.0, 38.3, 32.0, 26.5, 26.4, 26.1, 24.7, 22.4.

MS (Electrospray): 396.2 (M⁺+H).

### EXAMPLE 8

Synthesis of (1′S,2′S,5′R,6′S)-3-Allyloxycarbonyl-5-oxo-oxazolidinone-4-spiro-2′-bicyclo[3.1.0]hexane-6′-carboxylic acid 6′-benzoyloxymethyl ester.

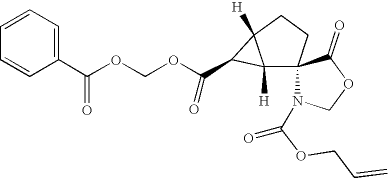

The title compound was prepared according to General Procedure 1 using chloromethyl benzoate (which was obtained as described by Davidsen et al. J. Med. Chem. 1994, 37, 4423-4429).

66% Yield. Colorless syrup.

[α]_{D}²⁵=+32.3° (c=1.5, CHCl₃).

¹H NMR (CDCl₃) δ: 8.02 (dd, 2H, J=8.8, 0.6 Hz), 7.59-7.52 (m, 1H), 7.40 (m, 2H), 5.98-5.79 (m, 1H), 5.97 (brs, 2H), 5.28 (brs, 2H), 5.25 (dd, 1H, J=17.0, 1.4 Hz), 5.19 (dd, 1H, J=10.4, 1.4 Hz), 4.57 (AB system, 2H), 2.58 (m, 1H), 2.35-2.18 (m, 2H), 2.10-1.92 (m, 3H), 1.73-1.62 (m, 1H).

¹³C NMR (CDCl₃) δ: 173.2, 171.0, 151.2, 133.5, 131.6, 129.8, 129.8, 128.8, 128.4, 128.3, 118.9, 79.6, 76.9, 66.4, 66.2, 32.4, 26.8, 26.3, 24.8, 22.6.

MS (Electrospray): 438.2 (M⁺+Na).

### EXAMPLE 9

Synthesis of (1′S,2′S,5′R,6′S)-3-Allyloxycarbonyl-5-oxo-oxazolidinone-4-spiro-2′-bicyclo[3.1.0]hexane-6′-carboxylic acid 6′-isopropoxycarbonyloxymethyl ester.

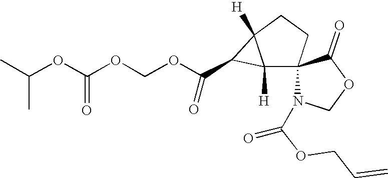

The title compound was prepared according to General Procedure 1 using chloromethyl isopropyl carbonate (which was obtained from chloromethyl chloroformate as described by Patonay, T. et al, Synth. Commun. 1990, 20, 2865-2885 and Davidsen et al. J. Med. Chem. 1994, 37, 4423-4429).

64% Yield. Colorless syrup.

[α]_{D}²⁵=+38.2 (c=1.1, CHCl₃).

¹H NMR (CDCl₃) δ: 5.94-5.80 (m, 1H), 5.63 (AB system, 2H), 5.26 (dd, 1H, J=15.6, 1.4 Hz), 5.25 (brs, 2H), 5.18 (dt, 1H, J=8.8, 1.2 Hz), 4.82 (m, 1H), 4.55 (m, 2H), 2.50 (m, 1H), 2.30-2.15 (m, 2H), 2.02-1.88 (m, 3H), 1.65 (m, 1H), 1.22 (d, 6H, J=6.2 Hz).

¹³C NMR (CDCl₃) δ: 173.1, 170.7, 153.0, 151.1, 131.6, 118.8, 81.6, 76.8, 72.7, 66.3, 66.1, 32.3, 26.8, 26.1, 24.8, 22.4, 21.3, 21.3.

### EXAMPLE 10

Synthesis of (1′S,2′S,5′R,6′S)-3-Allyloxycarbonyl-5-oxo-oxazolidinone-4-spiro-2′-bicyclo[3.1.0]hexane-6′-carboxylic acid 6′-n-butyryloxymethyl ester.

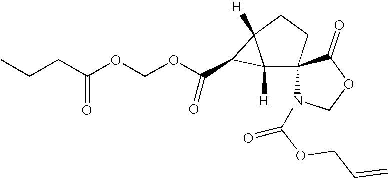

The title compound was prepared according to General Procedure 1 using chloromethyl n-butyrate.

51% Yield. Colorless syrup.

[α]_{D}²⁵=+44.4° (c=2.0, CHCl₃).

¹H NMR (CDCl₃) δ: 5.92 (m, 1H), 5.72 (AB system, 2H), 5.37-5.25 (m, 2H), 5.33 (brs, 2H), 4.62 (brd, 2H, J=6.0 Hz), 2.58 (brs, 1H), 2.37-2.31 (m, 4H), 2.10-1.97 (m, 3H), 1.73-1.56 (m, 3H), 0.94 (t, 3H, J=8.0 Hz). ³C NMR (CDCl₃) δ: 176.3, 173.7, 172.4, 154.1, 131.7, 119.2, 79.2, 76.9, 66.6, 66.3, 35.7, 32.4, 26.9, 26.4, 25.0, 22.7, 18.0, 13.4.

MS (Electrospray): 404.2 (M⁺+Na⁺).

### EXAMPLE 11

Synthesis of (1′S,2′S,5′R,6′S)-3-Allyloxycarbonyl-5-oxo-oxazolidinone-4-spiro-2′-bicyclo[3.1.0]hexane-6′-carboxylic acid 6′-methoxycarbonyloxymethyl ester

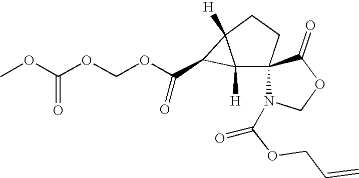

The title compound was prepared according to General Procedure 1 using chloromethyl methyl carbonate (which was obtained from chloromethyl chloroformate as described by Patonay, T. et al, Synth. Commun. 1990, 20, 2865-2885 and Davidsen et al. J. Med. Chem. 1994, 37, 4423-4429).

Colorless syrup.

¹H NMR (CDCl₃) δ: 5.93-5.78 (m, 1H), 5.72 (AB system, 2H), 5.40-5.25 (AB system 2H), 5.33 (brs, 2H), 4.65 (m, 2H), 3.84 (s, 3H), 2.68 (m, 1H), 2.50-2.25 (m, 2H), 2.22-1.90 (m, 3H), 1.55 (m, 1H).

### EXAMPLE 12

Synthesis of (1′S,2′S,5′R,6′S)-3-Allyloxycarbonyl-5-oxo-oxazolidine-4-spiro-2′-bicyclo[3.1.0]hexane-6′-carboxylic acid 6′-(5-methyl-2-oxo-[1,3]dioxolen-4-enyl)methyl ester.

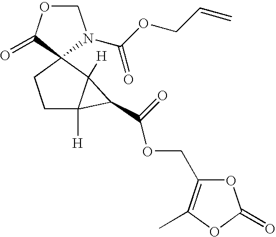

The title compound was prepared according to General Procedure 1 using 4-bromomethyl-5-methyl-[1,3]dioxol-2-one [Alexander, J, U.S. Pat. No. 5,466,811 (1995)]. Flash chromatography of the crude (hexanes/ethyl acetate 5:2) provided the ester. 64% yield. White foam.

¹H-NMR (CDCl₃) δ: 6.05-5.85 (m, 1H); 5.38-5.25 (m, 5H); 4.86 and 4.79 (AB system, J=14.5 Hz, 2H); 4.63 (dc, J=5.9, 1.1 Hz, 2H); 2.61 (m, 1H); 2.49-1.95 (m, 5H); 2.16 (s, 3H); 1.78-1.68 (m, 1H).

### General Procedure 2

General Procedure for 2-Allyloxycarbonylamino-6-allyl Ester and 2-Allyoxycarbonyl-oxazolidine Deprotection and Hydrochloride Formation.

The corresponding (1S,2S,5R,6S)-2-allyloxycarbonylamino-bicyclo[3.1.0]hexane-2,6-dicarboxylic acid 2-ester 6-allyl or 2-allyoxycarbonyl-oxazolidine ester was dissolved in dry dichloromethane (0.1 M solution) under nitrogen. 1,3-Dimethylbarbituric acid (3.0 equiv) and tetrakis(triphenylphosphine)palladium(0) (0.03 equiv) were added and the solution was heated at 35° C. for 2 h. After cooling to room temperature, the solvent was removed under vacuum and the resulting residue was dissolved in a solution of ethyl acetate saturated with hydrogen chloride gas and stirred for 2 h. In the case where a solid appeared, the reaction was filtered. The filtrated material was washed with ethyl acetate and ether, and dried to provide the product. In the case where a solid did not appear, the solvent was removed under vacuum, ethyl ether was added and the mixture was stirred overnight. The solid was filtered, washed thoroughly with ether and dried to provide the product.

### EXAMPLE 13

(1S,2S,5R,6S)-2-Amino-bicyclo[3.1.0]hexane-2,6-dicarboxylic acid 2-(3′-phthalidyl) ester hydrochloride

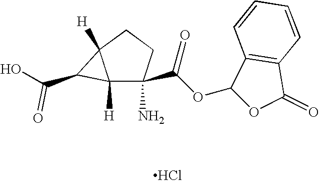

The title compound was prepared according to General Procedure 2.83% Yield. White solid. Mixture of diastereomers.

¹H NMR (Methanol-d₄) δ: (mixture of diastereomers): 7.98-7.60 (m, 4H), 7.59 (brs,1H), 2.38-2.21 (m, 2H), 2.17-2.04 (m, 4H), 1.66-1.58 (m, 1H).

¹³C NMR (Methanol-d₄) δ; (mixture of diastereomers): 174.4, 170.4, 170.3, 169.0, 145.0, 144.9, 136.6, 136.6, 133.0, 127.3, 127.3, 126.7, 126.7, 125.2, 125.1, 95.6, 95.5, 67.1, 66.9, 33.6, 33.6, 31.6, 31.4, 29.8, 29.8, 27.9, 27.8, 22.3, 22.3.

MS (Electrospray): 317.2 (M⁺+1).

### EXAMPLE 14

(1S,2S,5R,6S)-2-Amino-bicyclo[3.1.0]hexane-2,6-dicarboxylic acid 2-isopropyloxycarbonyloxymethyl ester hydrochloride

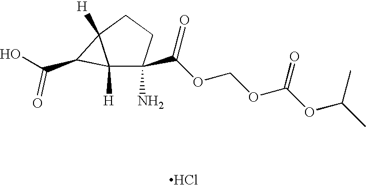

The title compound was prepared according to General Procedure 2.41% Yield. White solid. mp 59-60° C.

[α]_{D}²⁵=+11.4° (c=0.7, MeOH).

¹H NMR (Methanol-d₄) δ: 5.90 (brs, 2H), 4.88 (m, 1H), 2.33-2.01 (m, 6H), 1.62-1.55 (m, 1H), 1.31 (d, 6H, J=6.2 Hz).

¹³C NMR (Methanol-d₄) δ: 173.0, 168.8, 153.1, 83.0, 73.2, 65.5, 32.2, 29.9, 28.4, 26.3, 20.8, 20.3.

MS (Electrospray): 302.2 (M⁺+1).

### EXAMPLE 15

(1S,2S,5R,6S)-2-Amino-bicyclo[3.1.0]hexane-2,6-dicarboxylic acid 2-(4′-methoxy-benzoyloxy)methyl ester hydrochloride

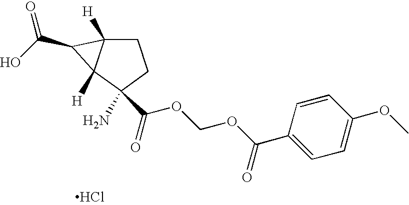

The title compound was prepared according to General Procedure 2. 51% Yield.

¹H-NMR (Methanol-d₄): 8.03 (dd, J=6.9, 2.0 Hz, 2H), 7.04 (dd, J=6.9, 2.1 Hz, 2H), 6.13 (q, J=5.1 Hz, 2H), 3.88 (s, 3H), 2.32-1.97 (m, 6H), 1.57-1.30 (m, 1H).

¹³C-NMR (Methanol-d₄): 174.5, 170.5, 166.0, 133.2, 121.8, 115.7, 115.2, 114.7, 82.4, 67.1, 56.2, 33.6, 31.5, 29.9, 27.8, 22.3.

### EXAMPLE 16

(1S,2S,5R,6S)-2-Allyloxycarbonylamino-bicyclo[3.1.0]hexane-2,6-dicarboxylic acid 2-(naphthalene-2′-carbonyloxymethyl) ester hydrochloride

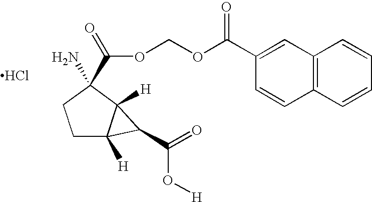

The title compound was prepared according to General Procedure 2. 66% yield. Solid. m.p. 184-185° C.

IR (KBr) ν: 3400-2400, 2978, 1769, 1734, 1684, 1599, 1578, 1541, 1447, 1410, 1283, 1203, 1076 cm⁻¹.

¹H NMR (Methanol-d₄) δ: 8.69 (brs, 1H), 8.02 (m, 4H), 7.63 (m, 2H), 6.23 (AB system, 2H), 2.42-1.99 (m, 6H), 1.56 (m, 1H).

¹³C NMR (Methanol-d₄) δ: 174.7, 170.6, 166.6, 137.6, 134.0, 133.0, 130.7, 130.3, 129.9, 129.1, 128.4, 127.2, 126.1, 82.7, 67.3, 33.8, 31.7, 30.1, 27.9, 22.4.

MS (Electrospray): 392.2 (M⁺+Na), 370.1 (M⁺+H).

### EXAMPLE 17

(1S,2S,5R,6S)-2-amino-bicyclo[3.1.0]hexane-2,6-dicarboxylic acid 2-(naphthalene-1′-carbonyloxymethyl) ester hydrochloride

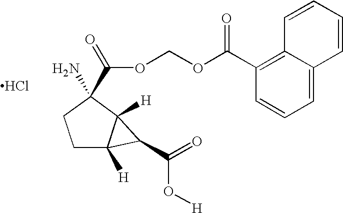

The title compound was prepared according to General Procedure 2. 91% yield. Solid. m.p. 212-213° C.

IR (KBr) ν: 3200-2400, 1767, 1732, 1682, 1581, 1531, 1512, 1447, 1373, 1333, 1273, 1244, 1205, 1107, 908 cm⁻¹.

¹H NMR (Methanol-d₄) δ: 8.87 (d, 1H), 8.28 (dd, 1H), 8.16 (d, 1H), 7.97 (m, 1H), 7.69-7.52 (m, 3H), 6.25 (s, 2H), 2.42-2.00 (m, 6H), 1.59 (m, 1H).

¹³C NMR (Methanol-d₄) δ: 174.7, 170.7, 167.0, 136.0, 135.6, 132.8, 132.4, 130.1, 129.5, 127.8, 126.4, 125.8, 82.6, 67.3, 33.9, 31.6, 30.1, 28.0, 22.5.

MS (Electrospray): 392.1 (M⁺+Na), 370.1 (M⁺+H).

### EXAMPLE 18

(1S,2S,5R,6S)-2-Amino-bicyclo[3.1.0]hexane-2,6-dicarboxylic acid 2-cyclohexyloxycarbonyloxymethyl ester

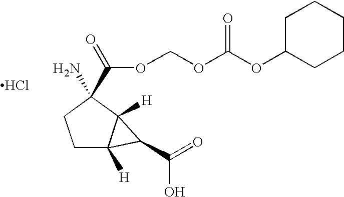

The title compound was prepared according to General Procedure 2. 63% yield. Solid.

IR (KBr) ν: 3500-2500, 2941, 2863, 1761, 1699, 1591, 1514, 1452, 1319, 1280, 1253, 1188, 1096 cm⁻¹.

¹H NMR (Methanol-d₄) δ: 5.90 (s, 2H,), 4.67 (m, 1H), 2.35-1.27 (m, 17H).

¹³C NMR (Methanol-d₄) δ: 174.7, 170.6, 154.8, 84.8, 79.5, 67.4, 33.9, 32.6, 31.7, 30.2, 28.1, 26.5, 24.7, 22.7.

MS (Electrospray): 364.1 (M⁺+Na), 342.1 (M⁺+H).

### EXAMPLE 19

Synthesis of (1S,2S,5R,6S)-2-Amino-bicyclo[3.1.0]hexane-2,6-dicarboxylic acid 6-tert-butylcarbonyloxymethyl ester hydrochloride

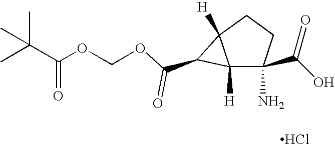

The title compound was prepared according to General Procedure 2 using (1′S,2′S,5′R,6′S)-3-allyloxycarbonyl-5-oxo-oxazolidinone-4-spiro-2′-bicyclo[3.1.0]hexane-6′-carboxylic acid 6′-(tert-butylcarbonyloxy)methyl ester.

74% Yield. White solid. mp 258° C. (dec.).

[α]_{D}²⁵=+13.7° (c=1.0, MeOH).

¹H NMR (Methanol-d₄) δ: 5.75 (brs; 2H), 2.32-2.05 (m, 6H), 1.58 (m, 1H), 1.19 (s, 9H).

¹³C NMR (Methanol-d₄) δ: 178.6, 172.6, 171.9, 81.2, 66.7, 39.7, 34.4, 31.2, 30.7, 27.7, 27.2, 22.0.

MS (Electrospray): 300.1 (M⁺+H).

### EXAMPLE 20

Synthesis of (1S,2S,5R,6S)-2-Amino-bicyclo[3.1.0]hexane-2,6-dicarboxylic acid 6-benzoyloxymethyl ester hydrochloride

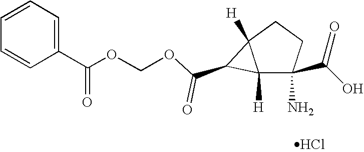

The title compound was prepared according to General Procedure 2 using (1′S,2′S,5′R,6′S)-3-allyloxycarbonyl-5-oxo-oxazolidinone-4-spiro-2′-bicyclo[3.1.0]hexane-6′-carboxylic acid 6′-(benzoyloxy)methyl ester.

32% Yield. White solid. mp 230° C., dec.

[α]_{D}²⁵+6.21° (c=0.66, MeOH).

¹H NMR (Methanol-d₄) δ: 8.03 (m, 2H), 7.64 (m, 1H), 7.54-7.46 (m, 2H), 6.00 (AB system, 2H), 2.38-2.05 (m, 6H), 1.59-1.48 (m, 1H).

¹³C NMR (Methanol-d₄) δ: 171.0, 170.3, 164.9, 133.6, 129.3, 128.7, 128.3, 79.6, 65.3, 33.1, 29.8, 29.2, 26.3, 20.5.

MS (Electrospray): 320.2 (M⁺+H), 342.1 (M⁺+Na⁺).

### General Procedure 3

General Procedure for 2-allyloxycarbonyl-oxazolidine deprotection and zwitterion formation.

The corresponding 2-allyloxycarbonyl-oxazolidine compound (1.0 equiv) was dissolved in dry dichloromethane (0.1 M solution) under nitrogen. 1,3-Dimethylbarbituric acid (3.0 equiv for each allyl group to be removed) and tetrakis (triphenylphosphine)palladium(0) (0.03 equiv) were added and the solution was heated at 35° C. for 2 h. After cooling to room temperature, the solvent was removed under vacuum and the resulting residue was dissolved in a solution of ethyl acetate saturated with hydrogen chloride gas and stirred for 2 h. In the case where a solid appeared, the reaction was filtered. The filtrated material was washed with ethyl acetate and ether, and dried to provide the product as the hydrochloride salt. In the case where a solid did not appear, the solvent was removed under vacuum, ethyl ether was added and the mixture was stirred overnight. The solid was filtered, washed thoroughly with ether and dried to provide the product as the hydrochloride salt. The hydrochloride salt was dissolved in the minimun ammount of dry methanol and filtered to remove any impurities. Propylene oxide (5 mL/mmol) was added to the homogeneous methanolic solution and the resulting mixture was stirred overnight. The precipitate was filtered, and washed with methanol and ethyl ether. Drying under high vacuum afforded the product as a zwitterion.

### EXAMPLE 21

Synthesis of (1S,2S,5R,6S)-2-Amino-bicyclo[3.1.0]hexane-2,6-dicarboxylic acid 6-tert-butylcarbonyloxymethyl ester.

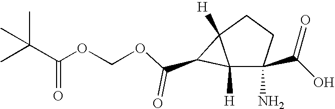

The title compound was prepared according to General Procedure 3 using (1′S,2′S,5′R,6′S)-3-allyloxycarbonyl-5-oxo-oxazolidinone-4-spiro-2′-bicyclo[3.1.0]hexane-6′-carboxylic acid 6′-tert-butylcarbonyloxymethyl ester.

46% Yield White solid. mp 205-206° C.

[α]_{D}²⁵=+21.3° (c=1.0, 1N HCl).

¹H NMR (D₂O/TFA-d₁) δ: 5.56 (AB system, 2H), 2.23 (m, 1H), 2.15-2.05 (m, 3H), 2.01-1.91 (m, 2H), 1.41 (m, 1H), 1.00 (s, 9H).

¹³C NMR (D₂O/TFA-d₄) δ: 179.2, 172.1, 171.6, 79.9, 65.4, 38.2, 32.6, 29.5, 29.4, 25.9, 25.6, 20.6.

MS (Electrospray): 300.3 (M⁺+H), 322.3 (M⁺+Na)

### EXAMPLE 22

Synthesis of (1S,2S,5R,6S)-2-Amino-bicyclo[3.1.0]hexane-2,6-dicarboxylic acid 6-benzoyloxymethyl ester.

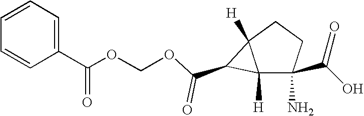

The title compound was prepared according to General Procedure 3 using (1′S,2′S,5′R,6′S)-3-allyloxycarbonyl-5-oxo-oxazolidinone-4-spiro-2′-bicyclo[3.1.0]hexane-61-carboxylic acid 6′-benzoyloxymethyl ester.

10% Yield. White solid. mp 210-212° C. (dec.)

[α]_{D}²⁵=+5.6° (c=0.25, 1N HCl).

¹H NMR (D₂O/TFA-d₁) δ: 7.86 (dd, 2H, J=7.2, 1.2 Hz), 7.50 (m, 1H), 7.36 (m, 2H), 5.83 (AB system, 2H), 2.29 (m, 1H), 2.13-1.89 (m, 5H), 1.45 (m, 1H).

### EXAMPLE 23

Synthesis of (1S,2S,5R,6S)-2-Amino-bicyclo[3.1.0]hexane-2,6-dicarboxylic acid 6-isopropyloxycarbonyloxymethyl ester.

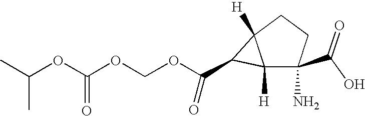

The title compound was prepared according to General Procedure 3 using (1′S,2′S,5′R,6′S)-3-allyloxycarbonyl-5-oxo-oxazolidinone-4-spiro-2′-bicyclo[3.1.0]hexane-6′-carboxylic acid 6′-isopropoxycarbonyloxymethyl ester.

36% Yield. White solid. mp 196-197° C.

[α]_{D}²⁵=+15.0° (c=0.18, 1N HCl).

¹H NMR (D₂O/TFA-d₁) δ: 5.49 (AB system, 2H), 4.67 (m, 1H), 2.19 (m, 1H), 2.11-1.80 (m, 4H), 1.86 (t, 1H, J=2.8 Hz), 1.33 (m, 1H), 1.04 (d, 6H, J=6.2 Hz).

¹³C NMR (D₂O/TFA-d₁) δ: 172.8, 171.9, 153.5, 82.2, 74.7, 65.5, 32.8, 29.7, 29.6, 25.9, 20.7, 20.4.

MS (Electrospray): 302.2 (M⁺+H), 324.1 (M⁺+Na⁺)

### EXAMPLE 24

Synthesis of (1S,2S,5R,6S)-2-amino-bicyclo[3.1.0]hexane-2,6-dicarboxylic acid 6-n-butyryloxymethyl ester.

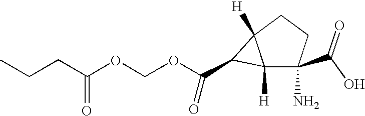

The title compound was prepared according to General Procedure 3 using (1′S,2′S,5′R,6′S)-3-allyloxycarbonyl-5-oxo-oxazolidinone-4-spiro-2′-bicyclo[3.1.0]hexane-6′-carboxylic acid 6′-(n-butyryloxymethyl) ester. 50% Yield. White solid. mp 199-200° C.

[α]_{D}²⁵=+10.8°(c=0.55, 1N HCl).

¹H NMR (D₂O/TFA-d₁) δ: 5.56 (AB system, 2H), 2.23 (t, 2H, J=7.2 Hz), 2.22-1.96 (m, 5H), 1.91 (t, 1H, J=3.0 Hz), 1.48-1.34 (m, 3H), 0.71 (t, 3H, J=7.1 Hz).

MS (Electrospray): 286.1 (M⁺+H), 308.21 (M⁺+Na⁺).

### EXAMPLE 25

Synthesis of (1S,2S,5R,6S)-2-amino-bicyclo[3.1.0]hexane-2,6-dicarboxylic acid 6-methoxycarbonyloxymethyl ester.

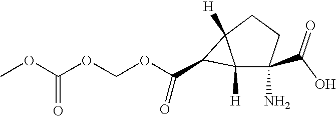

The tile compound was prepared according to General Procedure 3 using (1′S,2′S,5′R,6′S)-3-allyloxycarbonyl-5 oxo-oxazolidinone-4-spiro-2′-bicyclo[3.1.0]hexane-6′-carboxylic acid 6′-(methoxycarbonyloxy)methyl ester.

White solid. mp 214-215° C.

[α]_{D}²⁵=+17.3° (c=0.45, 1N HCl).

¹H NMR (D₂O/TFA-d₁) δ: 5.80 (AB system, 2H), 3.88 (s, 3H), 2.51-2.43 (m, 1H), 2.36-2.23-(m, 3H), 2.14 (t, 1H, J=3.0 Hz), 1.72 (m, 1H).

¹³C NMR (Methanol-d₄) δ: 171.5, 170.6, 155.0, 82.4, 65.8, 54.8, 33.6, 30.2, 29.7, 26.8, 21.0.

MS (Electrospray): 274.1 (M⁺+H), 296.1 (M⁺+Na⁺)

### EXAMPLE 26

Synthesis of (1S,2S,5R,6S)-2-amino-bicyclo[3.1.0]hexane-2,6-dicarboxylic acid 6-(5′-methyl-2′-oxo-[1′,3′]dioxolen-4′-yl)methyl ester.

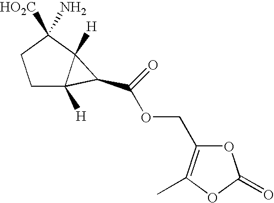

The tile compound was prepared according to General Procedure 3 using (1′S,2′S,5′R,6′S)-3-allyloxycarbonyl-5-oxo-oxazolidine-4-spiro-2′-bicyclo[3.1.0]hexane-6′-carboxylic acid 6′-(5-methyl-2-oxo-[1,3]dioxolen-4-enyl)methyl ester. The crude was purified by reverse phase chromatography using a C18 support eluting with mixtures of methanol and water. White solid. Yield 22%.

mp 180° C. (decomposition).

¹H-NMR (D₂O) δ: 4.95 (s, 2H); 2.29-2.00 (m, 5H); 2.13 (s, 3H); 1.94 (t, J=2.7 Hz, 1H); 1.50-1.35 (m, 1H).

[α]D₂₅=+5° (c=0.50, 1N HCl).

MS (Electrospray): 298 (M⁺+1).

### General Procedure 4

General Procedure for Diesterification of (1S,2S,5R,6S)-2-tert-Butoxycarbonylamino-bicyclo[3.1.0]hexane-2,6-dicarboxylic acid

(1S,2S,5R,6S)-2-tert-Butoxycarbonylamino-bicyclo[3.1.0]hexane-2,6-dicarboxylic acid (Example Preparation 2) (1.0 equiv) was dissolved in dry dimethylformamide (0.5 M solution) under nitrogen and potassium carbonate or cesium carbonate (2.0 equiv) was added at room temperature or 0° C. After 5 min, the corresponding halide (2.0 equiv) and sodium iodide (1.0 equiv) were added. The reaction was vigorously stirred at room temperature overnight. Water was added and the aqueous phase was extracted with portions of ethyl acetate. The combined organic extracts were washed with cold water, dried over magnesium sulfate and concentrated to dryness. The crude mixture was purified by silica gel flash-chromatography using the appropiate eluent (typically mixtures of hexanes/ethyl acetate).

### EXAMPLE 27

(1S,2S,5R,6S)-2-tert-Butoxycarbonylamino-bicyclo[3.1.0]hexane-2,6-dicarboxylic acid 2,6-dipivaloyloxymethyl ester

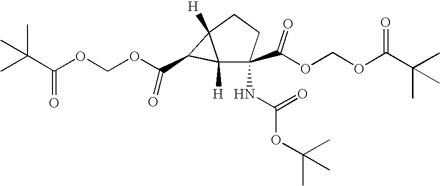

The title compound was prepared according to General Procedure 4 using commercially available chloromethyl pivalate.

71% Yield. Colorless syrup.

[α]_{D}²⁵=6.57° (c=1.4, CHCl₃).

¹H NMR (CDCl₃) δ: 5.82 (AB system, 2H), 5.74 (brs, 2H), 5.38 (brs, 1H), 2.46-2.32 (m, 2H), 2.20-1.91 (m, 3H), 1.76 (t, 1H, J=2.9 Hz), 1.42 (brs, 9H), 1.27 (m, 1H), 1.21 (s, 18H).

¹³C NMR (CDCl₃) δ: 177.0, 176.2, 171.5, 170.9, 155.1, 80.1, 79.6, 66.4, 38.7, 34.9, 32.4, 28.9, 28.2, 28.0, 26.8, 26.5, 20.8.

MS (Electrospray): 536.1 (M⁺+Na).

### EXAMPLE 28

(1S,2S,5R,6S)-2-tert-Butoxycarbonylamino-bicyclo[3.1.0]hexane-2,6-dicarboxylic acid 2,6-dibenzoyloxymethyl ester

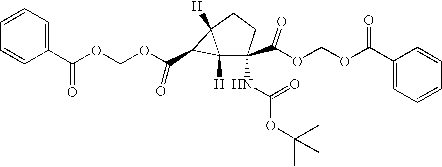

The title compound was prepared according to General Procedure 4 using chloromethyl benzoate (which was obtained as described by Davidsen et al. J. Med. Chem. 1994, 37, 4423-4429).

31% Yield. Colorless syrup.

[α]_{D}²⁵=−8.42° (c=1.08, CHCl₃).

¹H NMR (CDCl₃) δ: 8.03-7.97 (m, 4H), 7.57-7.48 (m, 2H), 7.42-7.33 (m, 4H), 6.01 (brs, 2H), 5.90 (brs, 2H), 5.55 (brs, 1H), 2.51-2.35 (m, 2H), 2.09-1.83 (m, 3H), 1.75 (t, 1H, J=2.7 Hz), 1.24 (m, 1H), 1.24 (brs, 9H).

¹³C NMR (CDCl₃) δ: 171.7, 171.0, 165.1, 165.0, 155.1, 133.6, 129.9, 129.6, 128.7, 128.4, 80.2, 79.4, 66.3, 34.9, 32.0, 29.0; 28.0, 26.3, 20.7.

MS (Electrospray): 576.1 (M⁺+Na).

### EXAMPLE 29

(1S,2S,5R,6S)-2-tert-Butoxycarbonylamino-bicyclo[3.1.0]hexane-2,6-dicarboxylic acid 2,6-diisopropyloxycarbonyloxymethyl ester

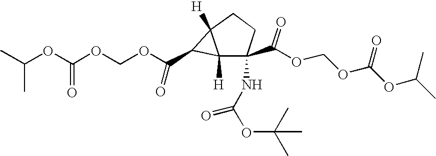

The title compound was prepared according to General Procedure 4 using chloromethyl isopropyl carbonate (which was obtained from chloromethyl chloroformate as described by Patonay, T. et al, Synth. Commun. 1990, 20, 2865-2885 and Davidsen et al. J. Med. Chem. 1994, 37, 4423-4429).

36% Yield. Colorless syrup.

[α]_{D}²⁵=−5.40° (c=1.0, CHCl₃).

¹H NMR (CDCl₃) δ: 5.81 (brs, 2H), 5.73 (brs, 2H), 5.37 (brs, 1H), 4.91 (m, 2H), 2.44 (m, 2H), 2.11-1.98 (m, 3H), 1.79 (t, 1H, J=2.8 Hz), 1.42 (brs, 9H), 1.34 (d, 6H, J=2.8 Hz), 1.30 (d, 6H, J=2.8 Hz), 1.28 (m, 1H).

¹³C NMR (CDCl₃) δ: 171.3, 170.8, 153.3, 153.2, 82.4, 81.6, 73.0, 72.9, 66.3, 34.5, 32.2, 29.0, 28.1, 26.5, 21.5, 21.5, 20.7.

MS (Electrospray): 540.1 (M⁺+Na).

### EXAMPLE 30

(1S,2S,5R,6S)-2-tert-Butoxycarbonylamino-bicyclo[3.1.0]hexane-2,6-dicarboxylic acid 2,6-di-(1′-isopropyloxycarbonyloxylethyl) ester

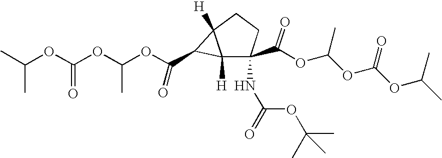

The title compound was prepared according to General Procedure 4 using 1-chloroethyl isopropyl carbonate (which was obtained from 1-chloroethyl chloroformate as described by Patonay, T. et al, Synth. Commun. 1990, 20, 2865-2885 and Davidsen et al. J. Med. Chem. 1994, 37, 4423-4429).

29% Yield. Cream syrup. Mixture of diastereomers.

¹H NMR (CDCl₃) δ (mixture of diastereomers): 6.83-6.68 (m, 2H), 5.09 (brs, 1H), 4.89 (m, 2H), 2.49-2.33 (m, 2H), 2.19-1.90 (m, 3H), 1.73-1.64 (m, 1H), 1.55-1.48 (m, 6H), 1.43 (brs, 9H), 1.32 (d, 6H, J=6.3 Hz), 1.29 (d, 6H, J=6.3 Hz), 1.26 (m, 1H).

¹³C NMR (CDCl₃) δ (mixture of diastereomers): 170.7, 170.3, 170.2, 155.0, 152.5, 92.1, 92.0, 91.2, 91.2, 72.8, 72.7, 66.5, 66.4, 35.0, 34.9, 32.4, 28.8, 28.5, 28.2, 27.0, 26.9, 26.5, 26.4, 21.2, 21.1, 21.0, 21.0, 20.9, 19.9, 19.5, 19.4.

MS (Electrospray): 568.2 (M⁺+Na).

### EXAMPLE 31

(1S,2S,5R,6S)-2-tert-Butoxycarbonylamino-bicyclo[3.1.0]hexane-2,6-dicarboxylic acid 2,6-di-(1′-ethoxycarbonyloxylethyl) ester

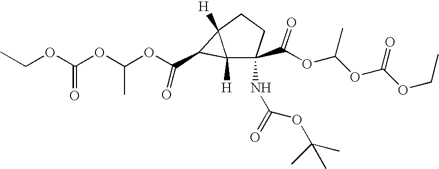

The title compound was prepared according to General Procedure 4 using commercially available 1-chloroethyl ethyl carbonate.

47% Yield. Cream syrup. Mixture of diastereomers.

¹H NMR (CDCl₃) δ (mixture of diastereomers): 6.84-6.65 (m, 2H), 5.21 (brs, 1H), 4.28-4.16 (m, 4H), 2.46 (m, 1H), 2.33 (m, 1H), 2.21-1.85 (m, 3H), 1.70 (m, 1H), 1.52-1.48 (m, 6H), 1.42 (s, 9H), 1.34-1.27 (m, 6H), 1.25 (m,1H).

¹³C NMR (CDCl₃) δ (mixture of diastereomers): 170.6, 170.2, 170.2, 170.1, 154.9, 152.8, 92.1, 92.0, 91.2, 91.2, 66.4, 66.3, 64.4, 64.3, 34.8, 32.2, 28.7, 28.5, 28.1, 26.8, 26.7, 26.3, 26.2, 21.0, 20.9, 20.8, 20.7, 19.4, 19.3, 14.0.

MS (Electrospray): 540.2 (M⁺+Na).

### EXAMPLE 32

(1S,2S,5R,6S)-2-tert-Butoxycarbonylamino-bicyclo[3.1.0]hexane-2,6-dicarboxylic acid 2,6-di-(3′-phthalidyl) ester

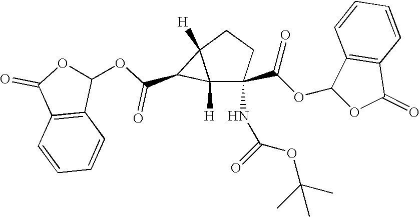

The title compound was prepared according to General Procedure 4 using 3-bromophthalide (which was obtained from phthalide as described by Koten et al. Org. Synth. Coll. Vol. V, 145-147).

89% Yield. White foamy solid.

¹H NMR (CDCl₃) δ (mixture of diastereomers): 7.92 (brd, 2H, J=7.0 Hz, 2H), 7.90-7.55 (m, 6H), 7.48 (brs, 1H), 7.39 (brs, 1H), 5.27-5.11 (br, 1H), 2.63-1.99 (m, 5H), 1.81-1.76 (m, 1H), 1.36 (brs, 9H), 1.28-1.21 (m, 1H).

¹³C NMR (CDCl₃) δ (mixture of diastereomers): 171.3, 170.8, 170.6, 167.6, 154.9, 144.0, 134.8, 131.2, 126.2, 126.2, 125.6, 125.6, 125.1, 124.2, 123.8, 123.5, 93.4, 92.7, 66.4, 35.0, 32.2, 29.4, 29.3, 29.0, 28.9, 28.0, 26.6, 26.0, 20.9, 20.7.

MS (Electrospray): 450.1 (M⁺+H-BOC), 572.2 (M⁺+Na)

### EXAMPLE 33

(1S,2S,5R,6S)-2-tert-Butoxycarbonylamino-bicyclo[3.1.0]hexane-2,6-dicarboxylic acid 2,6-dimethoxycarbonyloxymethyl ester.

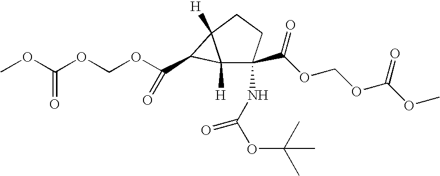

The title compound was prepared according to General Procedure 4 using chloromethyl methyl carbonate (which was obtained from chloromethyl chloroformate as described by Patonay, T. et al, Synth. Commun. 1990, 20, 2865-2885 and Davidsen et al. J. Med. Chem. 1994, 37, 4423-4429).

¹H NMR (CDCl₃) δ: 5.82 (s, 2H), 5.74 (s, 2H), 3.84 (s, 3H), 3.82 (s, 3H), 2.55-2.41 (m, 2H), 2.27-1.92 (m, 3H), 1.77 (t, 1H, J=3.0 Hz), 1.42 (s, 9H), 1.31-1.18 (m, 1H).

### EXAMPLE 34

(1S,2S,5R,6S)-2-tert-Butoxycarbonylamino-bicyclo[3.1.0]hexane-2,6-dicarboxylic acid 2,6-diisobutyryloxymethyl ester

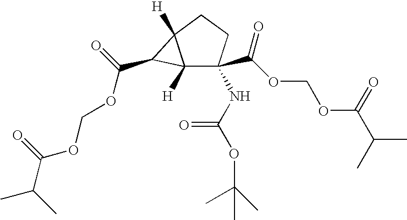

The title compound was prepared according to General Procedure 4 using chloromethyl isobutyrate (which was obtained using a similar procedure to the one described by Davidsen et al. J. Med. Chem. 1994, 37, 4423-4429). 44% Yield.

¹H-NMR (CDCl₃) δ: 5.80 (s, 2H), 5.71 (s, 2H), 5.14 (brs, 1H), 2.55 (sept., 2H, J=7.0 Hz), 2.39 (m, 2H), 2.05-1.90 (m, 3H), 1.74 (t, J=2.8 Hz, 1H), 1.41 (s, 9H), 1.28 (m, 1H), 1.20 (s, 6H), 1.16 (s, 6H).

¹³C-NMR (CDCl₃) δ: 175.2, 175.1, 172.7, 172.0, 155.1, 79.9, 79.3, 66.5, 34.9, 33.6, 32.3, 28.8, 28.1, 26.5, 20.8, 18.5.

### EXAMPLE 35

(1S,2S,5R,6S)-2-tert-Butoxycarbonylamino-bicyclo[3.1.0]hexane-2,6-di-(n-butyryloxymethyl) ester

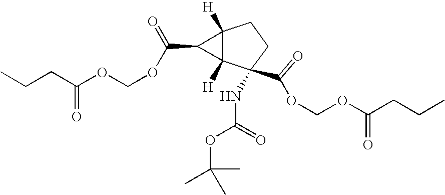

The title compound was prepared according to General Procedure 4 using commercially available chloromethyl butyrate.

71% yield. Oil.

IR (KBr) ν: 3389, 2974, 2940, 2880, 1761, 1717, 1506, 1456, 1423, 1368, 1254, 1169, 1105, 995 cm⁻¹.

¹H NMR (CDCl₃) δ: 5.77 (s, 2H), 5.69 (s, 2H), 5.31 (brs, 1H), 2.39 (m, 2H), 2.32 (t, 2H, J=7.4 Hz), 2.31 (t, 2H, J=7.4 Hz), 2.12-1.87 (m, 3H), 1.73 (t, J=2.8 Hz, 1H), 1.63 (st, 2H, J=7.4 Hz), 1.62 (st, 2H, J=7.4 Hz), 1.38 (s, 9H), 1.20 (m, 1H), 0.92 (t, 3H, J=7.4 Hz), 0.91 (t, 3H, J=7.4 Hz).

¹³C NMR (CDCl₃) δ: 172.2, 172.1, 171.6, 171.0, 155.0, 80.3, 79.7, 79.0, 66.2, 35.6, 34.9, 32.2, 28.9, 28.1, 26.5, 20.7, 18.0, 13.4.

MS (Electrospray): 508.2 (M⁺+Na).

### EXAMPLE 36

(1S,2S,5R,6S)-2-tert-Butoxycarbonylamino-bicyclo[3.1.0]hexane-2,6-di-(2′-naphthoyloxymethyl) ester

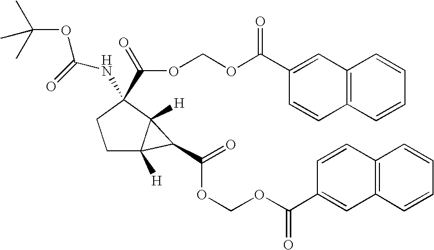

The title compound was prepared according to General Procedure 4 using chloromethyl 2-napthtoate (which was obtained using a similar procedure to the one described by Davidsen et al. J. Med. Chem. 1994, 37, 4423-4429). 59% yield. Oil.

IR (KBr) ν: 3382, 3061, 2980, 2937, 2874, 1736, 1630, 1599, 1508, 1471, 1423, 1390, 1368, 1282, 1153, 1128, 1082, 978 cm⁻¹.

¹H NMR (CDCl₃) δ: 8.63 (m, 2H), 8.05 (m, 2H), 7.93 (m, 2H), 7.85 (d, 4H), 7.56 (m, 4H), 6.12 (s, 2H), 6.02 (s, 2H), 5.34 (brs, 1H), 2.58-2.42 (m, 2H), 2.28-1.88 (m, 4H), 1.82 (t, 1H, J=2.8 Hz), 1.39 (s, 9H).

¹³C NMR (CDCl₃) δ: 171.7, 171.0, 165.3, 165.2, 155.0, 135.8, 132.3, 131.8, 129.4, 128.6, 128.2, 127.7, 126.7, 126.0, 125.2, 80.43, 79.6, 66.4, 35.0, 32.3, 29.0, 28.0, 26.5, 20.9.

MS (Electrospray): 676.1 (M⁺+Na).

### EXAMPLE 37

(1S,2S,5R,6S)-2-tert-Butoxycarbonylamino-bicyclo[3.1.0]hexane-2,6-di-(1′-naphthoyloxymethyl) ester

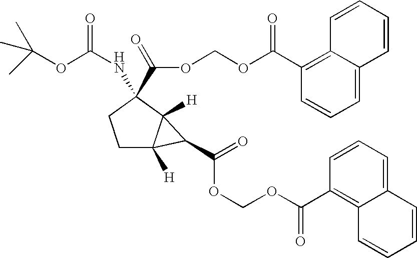

The title compound was prepared according to General Procedure 4 using chloromethyl 1-napthtoate (which was obtained using a similar procedure to the one described by Davidsen et al. J. Med. Chem. 1994, 37, 4423-4429).

63% yield. Oil.

IR (KBr) ν: 3387, 3053, 2980, 2937, 2874, 1732, 1593, 1576, 1510, 1456, 1423, 1392, 1368, 1332, 1278, 1240, 1159, 1115, 1001 cm⁻¹.

¹H NMR (CDCl₃) δ: 8.98 (t, 1H), 8.29 (m, 1H), 8.08 (d, 1H), 7.89 (d, 1H), 7.69-7.47 (m, 3H), 6.16 (s, 2H), 6.06 (s, 2H), 5.20 (br s, 1H), 2.60-2.45 (m, 2H), 2.30-1.92 (m, 3H), 1.83 (t, 1H, J=3.0 Hz), 1.31 (s, 9H), 1.29 (m, 1H).

¹³C NMR (CDCl₃) δ: 171.7, 171.1, 165.6, 155.1, 134.4, 134.3, 133.8, 131.6, 131.3, 128.6, 128.2, 128.1, 126.3, 125.6, 125.5, 125.1, 124.4, 80.4, 79.7, 66.5, 35.1, 32.4, 29.0, 28.1, 26.6, 20.9.

MS (Electrospray): 676.2 (M⁺+Na).

### EXAMPLE 38

(1S,2S,5R,6S)-2-tert-Butoxycarbonylamino-bicyclo[3.1.0]hexane-2,6-dicarboxylic acid dicyclohexyloxycarbonyloxymethyl ester

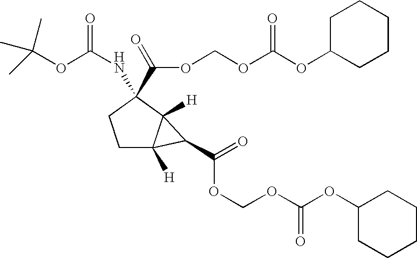

The title compound was prepared according to General Procedure 4 using chloromethyl cyclohexyl carbonate (which was obtained from chloromethyl chloroformate as described by Patonay, T. et al, Synth. Commun. 1990, 20, 2865-2885 and Davidsen et al. J. Med. Chem. 1994, 37, 4423-4429).

55% yield. Oil.

IR (KBr) ν: 3381, 2940, 2863, 1759, 1713, 1506, 1454, 1425, 1368, 1321, 1259, 1153, 1105 cm⁻¹.

¹H NMR (CDCl₃) δ: 5.80 (s, 2H), 5.72 (s, 2H), 5.21 (br s, 1H), 4.65 (m, 2H), 2.51-2.41 (m, 2H), 2.25-1.65 (m, 12H), 1.60-1.20 (m, 13H), 1.41 (s, 9H).

¹³C NMR (CDCl₃) δ: 171.3, 170.7, 155.0, 153.3, 153.2, 82.6, 81.8, 80.5, 77.8, 77.7, 66.4, 35.1, 32.3, 31.3, 31.2, 29.0, 28.1, 26.6, 25.1, 23.4, 20.8.

MS (Electrospray): 620.1 (M⁺+Na).

### EXAMPLE 39

(1S,2S,5R,6S)-2-tert-Butoxycarbonylamino-bicyclo[3.1.0]hexane-2,6-dicarboxylic acid 2,6-di-(1′-cyclohexyloxycarbonyloxy-ethyl) ester

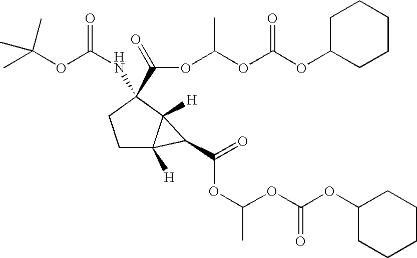

The title compound was prepared according to General Procedure 4 using 1-chloroethyl cyclohexyl carbonate (which was obtained from 1-chloroethyl chloroformate as described by Patonay, T. et al, Synth. Commun. 1990, 20, 2865-2885 and Davidsen et al. J. Med. Chem. 1994, 37, 4423-4429).

43% yield. Oil.

IR (KBr) ν: 3383, 2940, 2863, 1760, 1716, 1506, 1450, 1367, 1321, 1277, 1259, 1163, 1078 cm⁻¹.

¹H NMR (CDCl₃, mixture of 4 diastereomers) δ: 6.78 (q, 1H), 6.71 (q, 1H), 5.10 (brs, 1H), 4.63 (m, 2H), 2.50-2.33 (m, 2H), 2.22-1.64 (m, 12H), 1.60-1.20 (m, 19H), 1.42 (s, 9H).

¹³C NMR (CDCl₃, mixture of 4 diastereomers) δ: 170.6, 170.3, 170.2, 155.0, 152.4, 92.1, 92.0, 91.2, 80.3, 77.5, 77.4, 66.5, 66.4, 35.5, 34.9, 34.8, 32.3, 31.3, 28.8, 28.7, 28.5, 28.2, 27.0, 26.8, 26.4, 26.3, 25.1, 23.5, 21.1, 21.0, 20.9, 20.8, 19.5, 19.4.

MS (Electrospray): 648.1 (M⁺+Na).

### EXAMPLE 40

(1S,2S,5R,6S)-2-tert-Butoxycarbonylamino-bicyclo[3.1.0]hexane-2,6-dicarboxylic acid 2,6-diethoxycarbonyloxymethyl ester

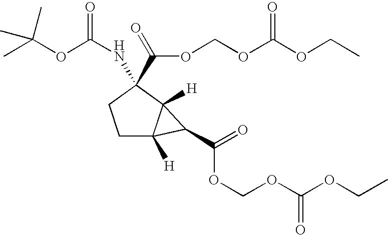

The title compound was prepared according to General Procedure 4 using chloromethyl ethyl carbonate (which was obtained from chloromethyl chloroformate as described by Patonay, T. et al, Synth. Commun. 1990., 20, 2865-2885 and Davidsen et al. J. Med. Chem. 1994, 37, 4423-4429).

70% yield. Oil.

IR (KBr) ν: 3389, 2984, 2941, 2876, 1763, 1713, 1506, 1456, 1425, 1392, 1369, 1263, 1153, 1001 cm¹.

¹H NMR (CDCl₃) δ: 5.80, 5.79 (s, 2H), 5.72, 5.71 (s, 2H), 5.28 (br s, 1H), 4.24, 4.23 (q, 2H), 4.22, 4.21 (q, 2H), 2.50-1.88 (m, 6H), 1.76 (m, 1H), 1.40 (s, 9H), 1.32, 1.31 (t, 3H), 1.30, 1.29 (t, 3H).

¹³C NMR (CDCl₃) δ: 171.3, 170.7; 155.0, 153.8, 153.7, 82.6, 81.8, 80.4, 66.4, 64.7, 35.1, 32.3, 29.0, 28.1, 26.5, 20.8, 14.0.

MS (Electrospray): 512.2 (M⁺+Na).

### EXAMPLE 41

(1S,2S,5R,6S)-Bis-(5-methyl-2-oxo-[1,3]dioxolen-4-ylmethyl) 2-tert-butoxycarbonylamino-bicyclo[3.1.0]hexane-2,6-dicarboxylate

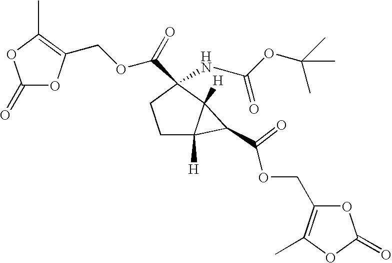

The title compound was prepared according to General Procedure 4 using cesium carbonate and 4-bromomethyl-5-methyl-[1,3]dioxolen-2-one (which was obtained as described in Alexander, J., U.S. Pat. No. 5,466,811, 1995).

34% yield as a white foam.

¹H-NMR (CDCl₃) δ: 5.25 (bs, 1H); 4.89 (s, 2H); 4.80 (s, 2H); 2.46-2.36 (m, 2H); 2.17 (s, 3H); 2.15 (s, 3H); 2.15-1.91 (m, 3H); 1.72 (t, J=2.9 Hz, 1H); 1.38 (s, 9H); 1.30-1.07 (m, 1H).

### General Procedure 5

General Procedure for 2-tert-Butoxycarbonylamino Deprotection and Hydrochloride Formation

The corresponding (1S,2S,5R,6S)-2-tert-butoxycarbonylamino-bicyclo[3.1.0]hexane-2,6-dicarboxylic ester was dissolved in a solution of ethyl acetate saturated with hydrogen chloride gas. The mixture was stirred at room temperature overnight. The reaction was evaporated to dryness. The resulting residue was dissolved in the minimum amount of ethyl acetate and triturated with diethyl ether. The precipitate formed was filtered, washed with ether and dried under vacuum to provide the product.

### EXAMPLE 42

(1S,2S,5R,6S)-2-Amino-bicyclo[3.1.0]hexane-2,6-dicarboxylic acid 2,6-dipivaloyloxymethyl ester hydrochloride

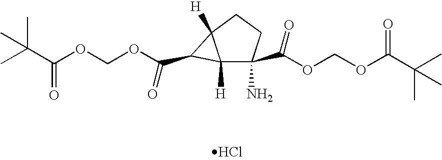

The title compound was prepared according to General Procedure 5. 84% Yield. Foamy white solid. mp 49-50° C.

[ ]D²⁵=+8.05° (c=0.41, MeOH).

¹H NMR (Methanol-d₄) δ: 5.92 (brs, 2H), 5.74 (brs, 2H), 2.27-2.17 (m, 6H), 1.62 (m, 1H), 1.23 (s, 9H), 1.19 (s, 9H).

¹³C NMR (Methanol-d₄) δ: 178.1, 175.1, 171.4, 170.0, 82.0, 81.1, 66.8, 39.8, 39.7, 34.1, 31.2, 30.6, 27.8, 27.4 (2 t-Bu), 22.1.

MS (Electrospray): 414.3 (M⁺+H).

### EXAMPLE 43

(1S,2S,5R,6S)-2-Amino-bicyclo[3.1.0]hexane-2,6-dicarboxylic acid, 2,6-dibenzoyloxymethyl ester hydrochloride

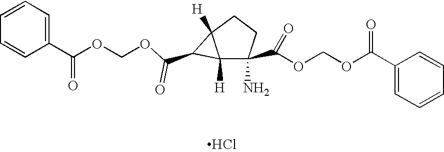

The title compound was prepared according to General Procedure 5.87% Yield. White foamy solid. mp 73° C.

[α]_{D}²⁵=−6.30° (c=1.0, MeOH).

¹H NMR (Methanol-d₄) δ: 8.09-8.00 (m, 4H), 7.72-7.63 (m, 2H), 7.57-7.47 (m, 4H), 6.16 (AB system, 2H), 5.99 (AB system, 2H), 2.38-2.01 (m, 6H), 1.55 (m, 1H).

¹³C NMR (Methanol-d₄) δ: 171.4, 170.2, 166.4, 166.3, 135.3, 135.1, 130.9, 130.8, 130.2, 129.9, 129.8, 129.5, 82.6, 81.2, 67.0, 34.2, 31.3, 30.6, 28.6, 27.7, 22.1.

MS (Electrospray): 454.3 (M⁺+H).

### EXAMPLE 44

(1S,2S,5R,6S)-2-Amino-bicyclo[3.1.0]hexane-2,6-dicarboxylic acid 2,6-diisopropyloxycarbonyloxymethyl ester hydrochloride

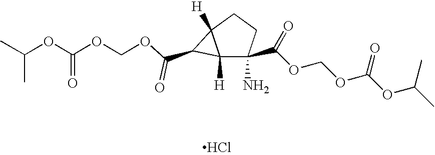

The title compound was prepared according to General Procedure 5.86% Yield. White foamy solid. mp 52° C.

[α]_{D}²⁵=+1.40° (c=1.0, MeOH).

¹H NMR (Methanol-d₄) δ: 5.91 (brs, 2H), 4.74 (AB system, 2H), 4.98-4.80 (m, 2H), 2.37-2.12 (m, 6H), 1.63 (m, 1H), 1.30 (d, 6H, J=6.2 Hz), 1.28 (d, 6H, J=6.2 Hz).

¹³C NMR (Methanol-d₄) δ: 171.3, 170.1, 154.8, 154.6, 84.6, 83.3, 74.7, 74.2, 67.0, 34.2, 31.2, 30.7, 27.8, 22.1, 21.9, 21.8.

MS (Electrospray): 418.2 (M⁺+H)

### EXAMPLE 45

(1S,2S,5R,6S)-2-Amino-bicyclo[3.1.0]hexane-2,6-dicarboxylic acid 2,6-di-(1′-isopropyloxycarbonyloxylethyl) ester hydrochloride

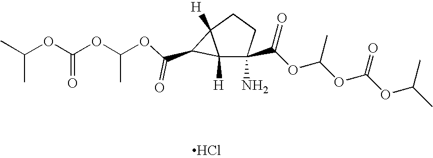

The title compound was prepared according to General Procedure 5.83% Yield. Foamy pale cream solid. Mixture of diastereomers.

¹H NMR (Methanol-d₄) δ (mixture of diastereomers): 6.82 (m, 1H), 6.71 (m, 1H), 4.88 (m, 2H), 2.33-2.05 (m, 6H), 1.62-1.57 (m, 3H), 1.60 (m, 1H), 1.50-1.47 (m, 3H), 1.34-1.23 (m, 12H).

¹³C NMR (Methanol-d₄) δ (mixture of diastereomers): 171.0, 171.0, 170.8, 169.7, 154.2, 154.2, 94.7, 94.5, 93.0, 93.0, 74.5, 74.0, 67.1, 34.4, 33.9, 31.5, 31.3, 30.7, 30.6, 30.5, 30.4, 30.1, 28.1, 28.0, 22.4, 22.3, 22.0, 22.0, 19.8, 19.6.

MS (Electrospray): 446.2 (M⁺+H), 469.2 (M⁺+Na).

### EXAMPLE 46

(1S,2S,5R,6S)-2-Amino-bicyclo[3.1.0]hexane-2,6-dicarboxylic acid 2,6-di-(1′-ethoxycarbonyloxylethyl) ester hydrochloride

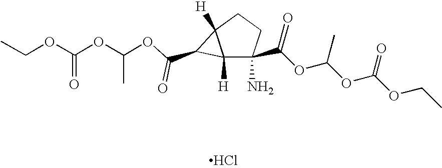

The title compound was prepared according to General Procedure 5.88% Yield. Foamy white solid.

¹H NMR (Methanol-d₄) δ (mixture of diastereomers): 6.81 (m, 1H), 6.72 (m, 1H), 4.28-4.12 (m, 4H), 2.33-2.07 (m, 6H), 1.61-1.55 (m, 3H), 1.58 (m, 1H), 1.49-1.46 (m, 3H), 1.34-1.23 (m, 12H).

¹³C NMR (Methanol-d₄) δ (mixture of diastereomers): 170.9, 170.8, 170.7, 169.5, 154.6, 154.5, 94.6, 94.5, 93.0, 67.0, 65.9, 65.5, 34.2, 33.8, 33.8, 31.4, 31.1, 30.6, 30.5, 30.4, 30.3, 27.9, 27.9, 27.8, 27.8, 22.2, 22.2, 22.1, 22.1, 19.6, 19.4, 14.4.

MS (Electrospray): 418.2 (M⁺+H), 440.2 (M⁺+Na).

### EXAMPLE 47

(1S,2S,5R,6S)-2-Amino-bicyclo[3.1.0]hexane-2,6-dicarboxylic acid 2,6-di-(3′-phthalidyl) ester hydrochloride

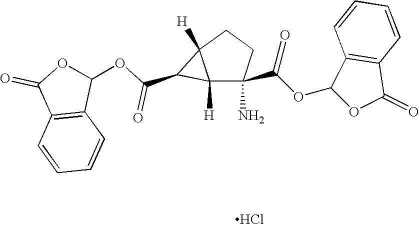

The title compound was prepared according to General Procedure 5.91% Yield. White solid. Mixture of diastereomers.

¹H NMR (Methanol-d₄) δ (mixture of diastereomers): 7.96-7.71 (m, 8H), 7.59 (brs,1H), 7.44 (brs, 1H), 2.56-2.12 (m, 6H), 1.71-1.62 (m, 1H)

¹³C NMR (Methanol-d₄) δ (mixture of diastereomers): 170.0, 169.8, 168.6, 168.6, 168.0, 167.5, 144.2, 143.4, 135.1, 134.8, 131.6, 131.2, 125.9, 125.8, 125.2, 124.9, 123.8, 123.7, 94.1, 93.1, 65.6, 60.1, 33.1, 33.0, 32.8, 32.7, 29.9, 29.7, 29.4, 29.3, 26.4, 20.7, 20.6.

MS (Electrospray): 450.1 (M⁺+H), 472.1 (M⁺+Na).

### EXAMPLE 48

(1S,2S,5R,6S)-2-Amino-bicyclo[3.1.0]hexane-2,6-dicarboxylic acid 2,6-dimethoxycarbonyloxymethyl ester hydrochloride

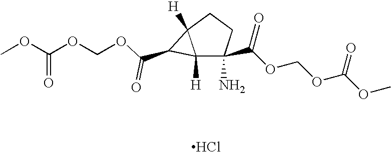

The title compound was prepared according to General Procedure 5. White solid mp 150° C., dec.

[α]_{D}²⁵=+14.2° (c 1.54, MeOH).

¹H NMR (Methanol-d₄) δ: 5.92 (AB system, 2H), 5.75 (AB system, 2H), 3.84 (s, 3H), 3.80 (s, 3H), 2.37-2.10 (m, 6H), 1.62-1.56 (m, 1H).

### EXAMPLE 49

(1S,2S,5R,6S)-2-Amino-bicyclo[3.1.0]hexane-2,6-dicarboxylic acid 2,6-diisobutyryloxymethyl ester hydrochloride

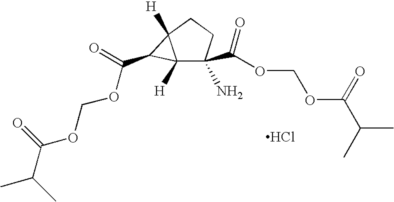

The title compound was prepared according to General Procedure 5.44% Yield. White solid. mp 224-226° C.

[α]_{D}²⁵=+26.3° (c=0.65, MeOH).

¹H-NMR (Methanol-d₄) δ: 5.91 (s, 2H), 5.74 (m, 2H), 2.57 (sept, J=7.0 Hz, 2H), 2.31-2.11 (m, 6H), 1.57 (m, 1H), 1.17 (t, J=6.8 Hz, 12H)

¹³C-NMR (Methanol-d₄) δ: 178.6, 178.3, 171.9, 169.4, 80.9, 80.2, 65.7, 33.6, 33.5, 32.8, 30.0, 29.7, 29.6, 29.2, 26.2, 21.0, 18.2, 17.8.

MS (Electrospray): 386.2 (M⁺+H).

### EXAMPLE 50

(1S,2S,5R,6S)-2-Amino-bicyclo[3.1.0]hexane-2,6-dicarboxylic acid 2,6-dipropanoyloxymethyl ester hydrochloride

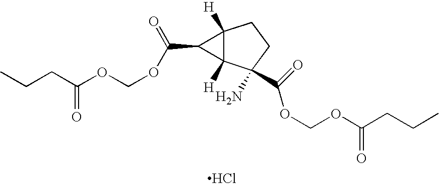

The title compound was prepared according to General Procedure 5.87% yield. Oil.

IR (KBr) ν: 3500-2500, 2973, 2880, 1770, 1589, 1520, 1462, 1427, 1342, 1263, 1157, 1101 cm⁻¹.

H NMR (Methanol-d₄) δ: 5.91 (s, 2H), 5.74 (s, 2H), 2.41 (t, 2H, J=7.2 Hz), 2.34 (t, 2H, J=7.2 Hz), 2.37-2.05 (m, 6H), 1.65 (st, 4H, J=7.2 Hz), 1.56 (m, 1H), 0.97 (t, 3H, J=7.2 Hz), 0.95 (t, 3H, J=7.2 Hz).

¹³C NMR (Methanol-d₄) δ: 173.7, 173.6, 171.6, 170.2, 82.0, 80.8, 67.0, 36.6, 36.5, 34.2, 31.4, 30.7, 27.9, 22.2, 19.3, 19.2, 14.0.

MS (Electrospray): 385.17.

### EXAMPLE 51

(1S,2S,5R,6S)-2-Amino-bicyclo[3.1.0]hexane-2,6-dicarboxylic acid 2,6-di-(2′-naphthoyloxymethyl) ester hydrochloride

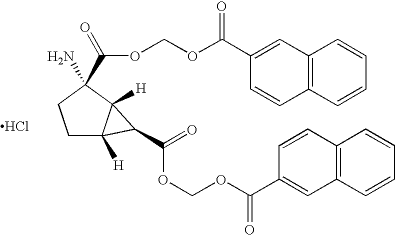

The title compound was prepared according to General Procedure 5.92% yield. Solid. m.p. 125-127° C.

IR (KBr) ν: 3500-2500, 2916, 2851, 1714, 1631, 1598, 1510, 1469, 1354, 1279, 1226, 1152, 1076 cm⁻¹.

¹H NMR (Methanol-d₄) δ: 8.62 (d, 2H), 7.95 (m, 8H), 7.60 (m, 4H), 6.21 (AB system, 2H), 6.04 (AB system, 2H), 2.43-2.01 (m, 6H), 1.57 (m, 1H).

¹³C NMR (Methanol-d₄) δ: 171.7, 170.4, 166.7, 166.6, 137.6, 137.5, 134.0, 133.0, 132.8, 130.6, 130.5, 130.3, 130.2, 129.8, 129.7, 129.0, 128.4, 128.3, 127.5, 127.1, 126.0, 82.8, 81.4, 67.2, 34.4, 31.5, 30.8, 27.9, 22.3.

MS (Electrospray): 554.1 (M⁺+H).

### EXAMPLE 52

(1S,2S,5R,6S)-2-Amino-bicyclo[3.1.0]hexane-2,6-dicarboxylic acid 2,6-di-(1′-naphthoyloxymethyl) ester hydrochloride

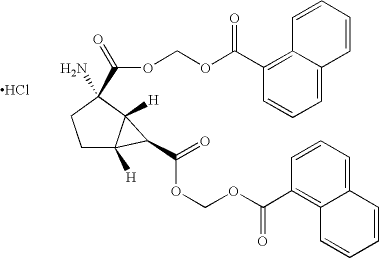

The title compound was prepared according to General Procedure 5.93% yield. Solid. m.p. 92-94° C.

IR (KBr) ν: 3407, 3054, 2947, 2880, 1732, 1593, 1578, 1512, 1462, 1427, 1346, 1317, 1278, 1242, 1169, 1113 cm⁻¹.

¹H NMR (Methanol-d₄) δ: 8.85 (m, 2H), 8.23 (m, 2H), 8.15 (m, 2H), 7.95 (m, 2H), 7.67-7.47 (m, 6H), 6.24 (AB system, 2H), 6.06 (AB system, 2H), 2.45-2.05 (m, 6H), 1.59 (m, 1H).

¹³C NMR (Methanol-d₄) δ: 171.7, 170.4, 167.0, 166.9, 136.0, 135.8, 135.5, 132.8, 132.4, 132.3, 130.1, 130.0, 129.5, 129.3, 127.8, 127.7, 126.5, 126.4, 126.2, 125.8, 125.7, 82.7, 81.4, 67.2, 34.4, 31.5, 30.8, 27.9, 22.3.

MS (Electrospray): 576.2 (M⁺+Na), 554.2 (M⁺+H).

### EXAMPLE 53

(1S,2S,5R,6S)-2-Amino-bicyclo[3.1.0]hexane-2,6-dicarboxylic acid 2,6-dicyclohexyloxycarbonyloxymethyl ester

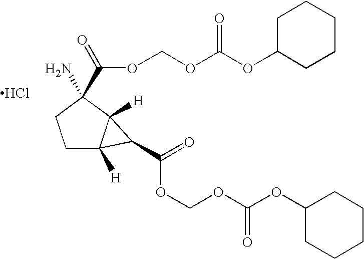

The title compound was prepared according to General Procedure 5.93% yield. Solid.

IR (KBr) ν: 3500-2500, 2941, 2863, 1759, 1585, 1520, 1454, 1427, 1319, 1253, 1157, 1097 cm⁻¹.

¹H NMR (Methanol-d₄) δ: 5.90 (AB system, 2H), 5.74 (AB system, 2H), 4.66 (m, 2H, 2 CH), 2.35-2.10 (m, 6H), 2.00-1.67 (m, 8H), 1.62-1.30 (m, 13H).

¹³C NMR (Methanol-d₄) δ: 171.4, 170.1, 154.8, 154.6, 84.9, 83.6, 79.3, 78.9, 67.0, 34.3, 32.5, 32.4, 31.5, 30.8, 28.0, 26.4, 24.6, 22.2.

MS (Electrospray): 498.2 (M⁺+H).

### EXAMPLE 54

(1S,2S,5R,6S)-2-Amino-bicyclo[3.1.0]hexane-2,6-dicarboxylic acid 2,6-di-(1′-cyclohexyloxycarbonyloxyethyl) ester hydrochloride

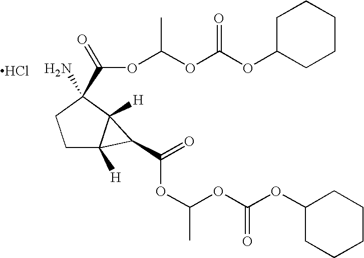

The title compound was prepared according to General Procedure 5.88% yield. Solid.

IR (KBr) ν: 3500-2500, 2941, 2863, 1759, 1585, 1518, 1450, 1427, 1379, 1359, 1317, 1256, 1190, 1078 cm⁻¹.

¹H NMR (Methanol-d₄, mixture of 4 diastereomers) δ: 6.81 (q, 1H), 6.69 (q, 1H), 4.61 (m, 2H), 2.40-1.20 (m, 33H).

¹³C NMR (Methanol-d₄, mixture of 4 diastereomers) δ: 171.0, 170.9, 170.3, 169.6, 154.2, 154.1, 94.6, 94.5, 93.0, 79.0, 78.5, 67.1, 34.5, 33.9, 32.5, 31.5, 31.2, 30.7, 30.6, 30.5, 30.4, 28.1, 28.0, 26.4, 24.6, 22.4, 22.3, 22.2, 19.8, 19.6.

MS (Electrospray): 548.2 (M⁺+Na), 526.2 (M⁺+H).

### EXAMPLE 55

(1S,2S,5R,6S)-2-Amino-bicyclo[3.1.0]hexane-2,6-dicarboxylic acid 2,6-diethoxycarbonyloxymethyl ester hydrochloride

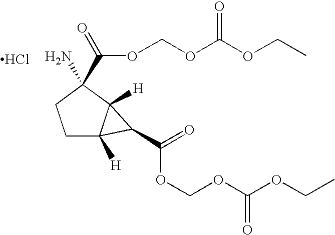

The title compound was prepared according to General Procedure 5.84% yield. Oil.

¹H NMR (CDCl₃) δ: 5.91 (m, AB system, 2H), 5.75 (m, AB system, 2H), 4.81 (brs, 1H), 4.26 (q, 2H), 4.21 (q, 2H), 2.40-2.10 (m, 6H), 1.63 (m, 1H), 1.31 (t, 3H), 1.29 (t, 3H).

¹³C NMR (CDCl₃) δ: 171.5, 170.2, 155.4, 155.3, 84.8, 83.5, 67.1, 66.3, 66.0, 34.3, 31.4, 30.8, 27.9, 22.2, 14.6.

MS (Electrospray): 412.1 (M⁺+Na).

### EXAMPLE 56

(1S,2S,5R,6S)-Bis-(5-methyl-2-oxo-[1,3]dioxolen-4-ylmethyl) 2-amino-bicyclo[3.1.0]hexane-2,6-dicarboxylate hydrochloride

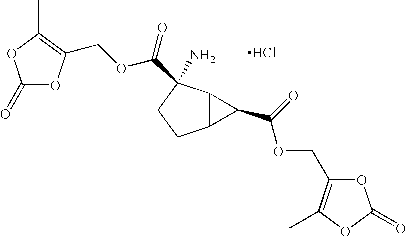

The title compound was prepared according to General Procedure 5.

60% yield as a white solid.

¹H-NMR (CDCl₃) δ: 5.14 (s, 2H); 4.93 (m, 2H); 2.27-2.08 (m, 6H); 2.19 (s, 3H); 2.13 (s, 3H); 1.54-1.44 (m, 1H).

¹³C-NMR (CDCl₃) δ: 171.2, 169.3, 152.2, 152.1, 141.2, 140.2, 133.3, 132.4, 66.3, 55.9, 54.2, 32.9, 30.1, 29.5, 26.6, 21.7, 9.3, 9.2.

### EXAMPLE 57

(1S,2S,5R,6S)-Bis-(5-methyl-2-oxo-[1,3]dioxolen-4-ylmethyl) 2-amino-bicyclo[3.1.0]hexane-2,6-dicarboxylate ethanesulfonic acid

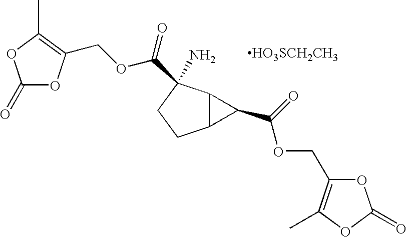

Trifluoroacetic acid (TFA) (9.07 mL, 118 mmol) was added to a solution of (1S,2S,5R,6S)-bis-(5-methyl-2-oxo-[1,3]dioxolen-4-ylmethyl) 2-tert-butoxycarbonylamino-bicyclo[3.1.0]hexane-2,6-dicarboxylate (6.00 g, 11.8 mmol, crude) in CH₂Cl₂ (60 mL) and stirred for 4 h. The resulting solution was partitioned between CH₂Cl₂ (50 mL) and H₂O (130 mL). An aqueous solution of NaHCO₃ (130 mL, 1.0 M) was then added. The phases were separated, and the aqueous phase was extracted with CH₂Cl₂ (50 mL). The combined organics were dried (MgSO₄) and filtered. Ethanesulfonic acid (EtSO₃H) (0.96 mL, 11.8 mmol) was then added followed by seed crystals. The solution was concentrated to a slurry (total weight of 60 g) and stirred at rt for 1 h. Diethyl ether (60 mL) was then added. The resulting slurry was stirred at rt for 2.5 h, filtered and dried under vacuum at 45° C. to afford 4.43 g (72%) of the title compound as a white crystalline solid.

[α]D=−1.92 (c 2.0, MeOH).

¹H NMR (CD₃OD) δ: 5.18 and 5.15 (AB quartet, JAB=13.8 Hz, 2H), 4.98 and 4.93 (AB quartet, JAB=13.8 Hz, 2H), 2.79 (q, J=7.5 Hz, 2H), 2.31-2.19 (m, 4H), 2.21 (s, 3H), 2.15 (s, 3H), 2.12-2.08 (m, 2H), 1.57-1.51 (m, 1H), 1.29 (t, J=7.5 Hz, 3H).

¹³C NMR (CD₃OD) δ: 172.2, 170.8, 153.7, 153.6, 142.7, 141.9, 135.0, 134.2, 67.1, 57.3, 55.5, 46.6, 34.0, 31.3, 30.3, 27.7, 22.1, 9.7, 9.2, 9.1.

LRMS (EI) calcd for C₁₈H₂₀NO₁₀ (M+−C₂H₅O₃S) 410.11, found 410.19 m/z.

1. A compound of the formula I 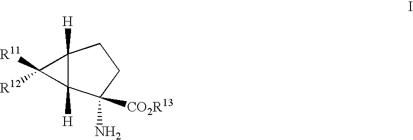
wherein
R¹¹ is CO₂R¹⁴ and R¹² is hydrogen or fluoro; or R¹¹ is hydrogen or fluoro and R¹² is CO₂R¹⁴;
R¹³ and R¹⁴ are, independently, hydrogen, —CHR¹⁵O₂CXR¹⁶ or a group selected from 3-phthalidyl or 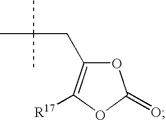
X is O, N, S, or a bond;
R¹⁵ is hydrogen, (1-10C) alkyl, (2-4C) alkenyl, aryl, or arylalkyl;
R¹⁶ is (1-10C) alkyl, (2-4C) alkenyl, (2-4C) alkynyl, or aryl; and
R¹⁷ is hydrogen, (1-10C) alkyl or phenyl;
provided when R¹⁴ is hydrogen, R¹³ is not hydrogen;
or a pharmaceuticaiiy acceptable salt tnereof.

2. A compound of claim 1 wherein
R¹¹ is CO₂R¹⁴ and R¹² is hydrogen or fluoro; or R¹¹ is hydrogen or fluoro and R¹² is CO₂R¹⁴;
R¹³ and R¹⁴ are, independently, hydrogen, —CHR¹⁵O₂CXR¹⁶ or a group selected from 3-phthalidyl or 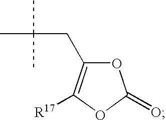
X is O, N, S, or a bond;
R¹⁵ is hydrogen, (1-10C) alkyl, (2-4C) alkenyl, aryl, or arylalkyl;
R¹⁶ is (1-10C) alkyl, (2-4C) alkenyl, (2-4C) alkynyl, or aryl; and
R¹⁷ is (1-1° C.) alkyl.

3. The compound (or salt thereof) of any one of claims 1 or 2 wherein (1-10C) alkyl is methyl, ethyl, n-propyl, isopropyl, t-butyl, or cyclohexyl; and
aryl is phenyl, 4-methoxyphenyl, naphthan-1-yl or naphthan-2-yl.

4. The compound (or salt thereof) of any one of claims 1 or 2 wherein
R¹¹ is CO₂R¹⁴;
R¹² is hydrogen;
R¹³ and R¹⁴ are, independently, hydrogen, —CHR¹⁵O₂CXR¹⁶, or a group selected from 3-phthalidyl or 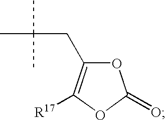
R¹⁵ is hydrogen or methyl;
R¹⁶ is isopropyl, 4-methoxyphenyl, phenyl, napthalen-1-yl, napthalen-2-yl, cyclohexyl, t-butyl, n-propyl, methyl, or ethyl;
R¹⁷ is methyl; and
X is O or a bond.

5. The compound (or salt thereof) of any one of claims 1 or 2 wherein R¹³ and R¹⁴ are —CHR¹⁵O₂CXR¹⁶ or a group selected from 3-phthalidyl or

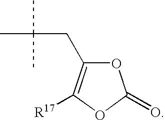

6. The compound (or salt thereof) of claim 5 which is selected from:
a) (1S,2S,5R,6S)-2-amino-bicyclo[3.1.0]hexane-2,6-dicarboxylic acid 2,6-dipivaloyloxymethyl ester hydrochloride;
b) (1S,2S,5R,6S)-bis-(5-methyl-2-oxo-[1,3]dioxolen-4-ylmethyl) 2-amino-bicyclo[3.1.0]hexane-2,6-dicarboxylate ethanesulfonic acid;
c) (1S,2S,5R,6S)-2-Amino-bicyclo[3.1.0]hexane-2,6-dicarboxylic acid 2,6-di-(3′-phthalidyl) ester hydrochloride; or
d) (1S,2S,5R,6S)-2-Amino-bicyclo[3.1.0]hexane-2,6-dicarboxylic acid 2,6-dipropanoyloxymethyl ester hydrochloride.

7. The compound (or salt thereof) of any one of claims 1 or 2 wherein R¹³ is —CHR¹⁵O₂CXR¹⁶ and R¹⁴ is hydrogen.

8. The compound (or salt thereof) of claim[[s 1-4 or]] 7 which is selected from:
a) (1S,2S,5R,6S)-2-Amino-bicyclo[3.1.0]hexane-2,6-dicarboxylic acid 2-(4′-methoxy-benzoyloxy)methyl ester hydrochloride; or
c) (1S,2S,5R,6S)-2-amino-bicyclo[3.1.0]hexane-2,6-dicarboxylic acid 2-(naphthalene-1′-carbonyloxymethyl) ester hydrochloride.

9. A pharmaceutically acceptable salt of a compound of formula I as claimed in any one of claims 1 or 2 which is an acid-addition salt made with an acid which provides a pharmaceutically acceptable anion or, for a compound which contains an acidic moiety, which is a salt made with a base which provides a pharmaceutically acceptable anion.

10. A pharmaceutical formulation comprising in association with a pharmaceutically acceptable carrier, dilutant or excipient, a compound of formula I (or a pharmaceutically acceptable salt thereof) as provided in any one of claims 1 or 2.

11. A process for preparing the compound of formula I, or a pharmaceutically acceptable salt thereof, as claimed in claim 1 comprising:
(A) for a compound of formula I in which R¹³ is an ester and R¹⁴ is hydrogen, deprotecting the amine group and selectively deprotecting the ester group of a compound of formula II 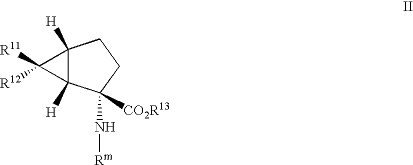
in which R¹³ is an ester and R¹⁴ is a carboxy protecting group;
(B) for a compound of formula II in which R¹³ and R¹⁴ are not hydrogen (a di-ester), esterifying a compound of formula II in which R¹³ is hydrogen and R¹⁴ is not hydrogen;
(C) for a compound of formula II in which R¹³ is hydrogen and R¹⁴ is not hydrogen, selectively esterifying a compound of formula II in which R¹³ and R¹⁴ are both hydrogen (a di-acid);
(D) for a compound of formula II in which R¹³ and R¹⁴ are both hydrogen (a di-acid), amidating the amine group of a compound of formula I with an amine protecting group;
(E) for a compound of formula I where R¹³ is hydrogen and R¹⁴ is an ester group, deprotecting and ring-opening a compound of formula IV 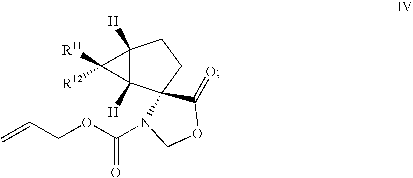
(F) for a compound of formula IV in which R¹⁴ is an ester group, esterifying the acid group of a compound of formula IV in which R¹⁴ is hydrogen;
(G) for a compound of formula IV in which R¹⁴ is hydrogen (an acid), protecting and cyclizing a compound of formula III;
(H) for a compound of formula I in which R¹³ and R¹⁴ are not hydrogen (a di-ester), deprotecting the amine group of a compound of formula II;
(I) for a compound of formula II in which R¹³ and R¹⁴ are not hydrogen (a di-ester), esterifying the carboxy groups of a compound of formula II in which R¹³ and R¹⁴ are both hydrogen (a di-acid);
whereafter, for any of the above procedures, when a functional group is protected using a protecting group, removing the protecting group;
whereafter, for any of the above procedures, when a pharmaceutically acceptable salt of a compound of formula I is required, it is obtained by reacting the basic form of such a compound of formula I with an acid affording a physiologically acceptable counterion, or, for a compound of formula I which bears an acidic moiety, reacting the acidic form of such a compound of formula I with a base which affords a pharmaceutically acceptable cation, or by any other conventional procedure.

12. (Cancelled)

13. (Cancelled)

14. A method for treating a neurological disorder in a patient which comprises administering to the patient in need of treatment thereof a pharmaceutically-effective amount of a compound of claim 1.

15. The method of claim 14 wherein said neurological disorder is cerebral deficits subsequent to cardiac bypass and grafting; cerebral ischemia; spinal cord trauma; head trauma; Alzheimer's Disease; Huntington's Chorea; amyotrophic lateral sclerosis; AIDS-induced dementia; perinatal hypoxia;
hypoglycemic neuronal damage; ocular damage and retinopathy; cognitive disorders;
idiopathic and drug-induced Parkinson's Disease; muscular spasms; migraine headaches; urinary incontinence; drug tolerance, withdrawal, and cessation; smoking cessation; emesis; brain edema; chronic pain; sleep disorders; convulsions; Tourette's syndrome; attention deficit disorder; and tardive dyskinesia.

16. The method of claim 15 wherein said neurological disorder is drug tolerance, withdrawal, and cessation; or smoking cessation.

17. (Cancelled)

18. (Cancelled)

19. (Cancelled)

20. A method for treating a psychiatric disorder in a patient which comprises administering to the patient in need of treatment thereof a pharmaceutically-effective amount of a compound of claim 1.

21. The method of claim 20 wherein said psychiatric disorder is schizophrenia, anxiety and related disorders, depression, bipolar disorders, psychosis, and obsessive compulsive disorders.

22. The method of claim 21 wherein said psychiatric disorder is anxiety and related disorders.

23. (Cancelled)

24. (Cancelled)

25. (Cancelled)

26. A pharmaceutical formulation comprising the compound of claim 1 in combination with one or more pharmaceutically-acceptable carriers, diluents, or excipients.

27. (Cancelled)

28. (Cancelled)
